(19) United States
(12) Patent Application Publication
(10) Pub. No.: US 2007/0282151 A1
(43) Pub. Date: Dec. 6, 2007

(54) Manufacture of higher hydrocarbons from methane, via methanesulfonic acid, sulfene, and other pathways

(76) Inventor: Alan K. Richards, Houston, TX (US)

Correspondence Address: Patrick D. Kelly, 11939 Manchester #403, St. Louis, MO, 63131, US

(21) Appl. No.: 11/438,103

(22) Filed: May 19, 2006

(51) Int. Cl. C07C 43/00; C07C 1/00

(52) U.S. Cl. 585/733; 585/638; 568/618

(57) ABSTRACT

Hydrocarbon liquids and olefins can be made from methane with greater efficiency than previously available, by converting methane into methanesulfonic acid (MSA), then converting the MSA into a reactive anhydride called sulfene, H₂C═SO₂. Sulfene will exothermically form ethylene, an olefin. It also can insert methylene groups (—CH₂—) into hydrocarbon liquids, to make heavier and more valuable liquids. Other options are disclosed for improved methods of making MSA (such as by using di(methyl-sulfonyl) peroxide as a radical initiator), for converting MSA into products such as dimethyl ether (DME), and for using DME as a “peak shaving” gas that can be injected into natural gas supply pipelines with no disruptions to end-use burners.

### BACKGROUND OF THE INVENTION

This invention relates to organic chemistry, hydrocarbon chemistry, and processing of methane gas.

Because there have been no adequate methods for converting methane gas into liquids that can be transported efficiently to commercial markets, huge volumes of methane are wasted every day, mainly by flaring or reinjection, at fields that produce crude oil. In addition, numerous gas fields are simply shut in, at numerous locations around the world.

Skilled chemists have tried for at least 100 years to develop methods for converting methane gas into various types of liquids. While various efforts in the prior art could produce relatively small quantities and low yields of methanol or other liquids, none of those efforts ever created yields that were sufficient to support commercial use at oil-producing sites. Such efforts prior to 1990 are described in reviews such as Gesser et al 1985 and Olah 1987 (full citations to all articles and books are provided below), and efforts after 1990 are described in articles such as Periana et al 1993, 1998, and 2002, Basickes et al 1996, Lobree et al 2001, and Mukhopadhyay 2002 and 2003.

As a result, oil and chemical companies are making huge investments in liquified natural gas (LNG) facilities, and in a processing system known as “Fischer-Tropsch”, to convert methane into liquids. However, both of those systems are very inefficient and wasteful.

LNG processing burns about 40% of a methane stream, to refrigerate the remainder to somewhere between −260 and −330° F., causing it to liquefy so it can be loaded into specialized ocean-going tankers. After a tanker reaches its destination, another large portion of the methane must be burned, to warm the remainder back up to temperatures that allow it to be handled by normal pipes and pumps. Therefore, LNG wastes roughly half of a methane stream. Nevertheless, as of mid-2006, oil companies had committed tens of billions of dollars to build LNG facilities.

Fischer-Tropsch processing burns about 30% of a methane stream, to convert the remainder into a carbon monoxide and hydrogen mix called “synthetic gas” or “syngas”. The syngas is then converted (using expensive catalysts) into heavy oils and paraffins, which then must be cracked and/or distilled to convert them into diesel fuel, heating oil, and other products. The syngas conversion, the catalyst costs, and the requirement for cracking thick and heavy oils and waxes all create inefficiencies, but as of mid-2006, companies have committed tens of billions of dollars to build Fischer-Tropsch facilities.

The wastes and inefficiencies of LNG and Fischer-Tropsch systems prove the assertion that any methane-to-methanol systems previously proposed, based on small-scale laboratory work, have not been regarded as commercially practical, by any major companies. In addition, most methanol conversion systems proposed to date generate large quantities of acidic and hazardous byproducts and toxic wastes. Even if they can be recycled, byproducts and wastes are major obstacles to efficient and economic use.

A processing system that enables much more efficient conversion of methane (and other light hydrocarbons), into methanol (and other stable liquids) is described in two Patent Cooperation Treaty (PCT) applications by the same inventor herein. The first PCT application, PCT/US03/035396 (filed in November 2003, published in May 2004 as WO 2004/041399) describes the use of a “radical initiator” to initiate a chain reaction, which will bond methane (CH₄) and sulfur trioxide (SO₃) to each other in a way that generates methane-sulfonic acid (H₃CSO₃H, abbreviated as MSA). Briefly, this process is commenced by using any of various known methods to remove an entire hydrogen atom (both a proton and an electron) from methane. This generates methyl “radicals”, with each radical having an unpaired electron, indicated as H₃C*. Several methods for carrying out this particular step (as described in previously-published patent applications) are illustrated in FIG. 1. More information about such methods is available in, for example, U.S. Pat. Nos. 3,927,189 (Jayawant 1975), 6,200,440 (Moran et al 2001) and 6,503,386 (Lehmann et al 2003), which relate to Marshall's acid, one of the main candidate initiators. Other articles, such as Danon et al 1987, Peng et al 1992, Chuang et al 1999, Romm et al 2001, Schwarz-Selinger et al 2001, Blavins et al 2001, and Zhai et al 2004) also should be consulted, because they describe devices that are often called “radical pumps” or “radical guns”, mostly involving nozzles that treat a liquid with heat or with UV or laser radiation as the liquid passes through a specialized treating nozzle.

When a strong radical initiator is mixed with methane gas, it will rapidly and efficiently remove hydrogen atoms from the methane, thereby creating methyl radicals. When the methyl radicals contact sulfur trioxide under suitable conditions in a reactor, the methyl radicals are not strong enough to remove hydrogen atoms from the SO₃. Therefore, the methyl radicals bond to the SO₃, in a way that creates larger and heavier radicals, having the formula H₃CSO₃*, as shown in FIG. 2. These radicals have enough strength to remove hydrogen atoms from fresh methane that is being pumped into the reactor vessel. When this happens, two things occur: (1) the H₃CSO₃* radicals are converted into MSA, a relatively stable liquid; and, (2) a new supply of methyl radicals is created, in a way that creates and sustains a chain reaction that leads to the creation of more MSA.

In addition to continuously forming MSA as a product, liquid MSA in the reactor vessel also acts as an “amphoteric” solvent (i.e., a solvent having two domains with different traits). The methyl domain of MSA helps methane gas dissolve and mix rapidly in the solution, while the sulfonic domain helps liquid SO₃ mix rapidly in the solution.

Liquid MSA, which is being formed in the reactor, is continuously removed from the outlet of the reactor. It can be used in several ways (such as in electroplating and semiconductor manufacturing), but it has only small and limited markets. Therefore, any of several alternate products can be created by using MSA as an intermediate. For example, it can be heated over a catalyst, in a manner that causes the MSA to “crack” in a way that releases methanol (H₃COH) and sulfur dioxide. Methanol (also called methyl alcohol) is a stable liquid that can be stored, shipped, and used as a chemical feedstock, liquid fuel, or fuel additive, and since it is a clean-burning fuel, it has virtually unlimited markets. The sulfur dioxide can be oxidized back to sulfur trioxide, which can be recycled back into the reactor vessel that is creating the MSA. Alternately, it can be processed in any of various ways, including various ways that make good and efficient use of the direct carbon-sulfur bond in MSA. Several basic types of “downstream” processing that can allow MSA to be used to manufacture chemicals (such as alkylamines, formaldehyde, aromatic compounds, dimethyl ether, etc.) are described in PCT application, PCT/US04/019977, filed in June 2004. It was published as WO 2005/069751 in August 2005, which was less than a year before the filing of this application; accordingly, it is not conceded to be prior art against the invention disclosed herein.

The radical-initiated chain reaction system disclosed in those PCT applications, and illustrated in FIGS. 1 and 2, was never developed by anyone prior to the Applicant, because prior researchers believed that any pathway that uses methyl radicals, which are extremely reactive and exceptionally difficult to control, will create low-value mixtures, having desired product molecules at relatively low concentrations, mixed with high concentrations of other undesired compounds. This would create severe processing and purification problems, which would render any such system unworkable, uneconomic, and unfeasible, when scaled up to the huge quantities that are involved in oil and gas processing. That belief, under the prior art, is described in articles such as Periana et al 1993. Accordingly, although chemistry professors at several universities were testing salt-and-acid mixtures (e.g., Basickes et al 1996, Lobree et al 2001, and Mukhopadhyay et al 2002, 2003, and 2004), using peroxy-acid salts such as K₂S₂O₈ (a potassium salt of a compound known as Marshall's acid), that work was regarded by industry as impractical and unfeasible, when scaled up to the huge volumes of methane gas that must be processed at large oil or gas fields. That perspective is set forth in articles such as Golombok et al 2003, which contains statements such as, “we never examined what happened to K₂S₂O₈, which one would never use in an industrial process.” Therefore, industrial attention focused on liquified natural gas (LNG) systems and “Fischer-Tropsch” systems, as described above. However, the inventor herein discovered that if a reactor system keeps the number of reagents to an absolute minimum, while uses a radical-initiated chain reaction to create MSA, the newly-developed system can convert methane into MSA with yields and selectivities that are believed to exceed 90%, and that appear capable of exceeding 95% or even 98%, when properly optimized.

Because of the role MSA plays in this system, and because MSA is a stable intermediate that will be transferred to other vessels for storage, processing, or other uses once it emerges from the MSA-forming reactor vessel, the terms “upstream” and “downstream” are used to divide various processing steps into two categories. Any references herein to “upstream” processing refer to steps that are involved in creating and/or purifying MSA, from methane and sulfur trioxide; this includes any steps used to create methyl radicals or any other radicals that can help initiate or sustain the chain reaction that will form the MSA. By contrast, references to “downstream” processing refer to steps involved in converting MSA into something else, after the MSA has emerged from a MSA-forming reactor vessel.

It also should be noted that the oxidation of SO₂ back to SO₃ is an accompanying and essential process, but it is neither upstream or downstream; instead, it will be part of a closed cycle that will be repeated endlessly, in ways that will enable and help drive the organic reactions. Oxygen-rich SO₃ will enter a reactor vessel, and in nearly all cases it will transfer and donate one of its oxygen atoms to an organic compound, using any of several pathways (the complete pathway will depend partly on how the MSA is processed into other products). In most such cases, once the SO₃ surrenders an oxygen atom, the depleted SO₂ (usually in gaseous form) will need to be oxidized back into SO₃, which can be pumped back into the MSA-forming reactor, to complete the closed cycle.

It should be noted that MSA does not need to be cracked, to release methanol, to accomplish the goal of converting methane gas into a stable and transportable liquid. Instead, as described in the published PCT applications cited above, it is also possible to process MSA directly on Zeolite, SAPO, or other porous catalysts, to directly create various other compounds, without passing through methanol as an intermediate.

The contents of both of the published PCT applications cited above are incorporated herein by reference, as though fully set forth herein.

For convenience, the MSA intermediate is regarded as the dividing point between “upstream” and “downstream” processing. Any steps, reactors, or devices used to make or purify MSA (or its precursors, such as Marshall's acid or any other radical initiator), or to keep an MSA-forming reactor running properly, are regarded as being on the upstream side of an operation. By contrast, any steps, reactors, or devices that receive MSA as a feedstock, intermediate, or product are on the “downstream” side of an operation. The MSA-forming reactor is analogous to a dam on a river; it is neither upstream nor downstream, and instead is what creates and defines the different upstream and downstream zones. The oxidation of SO₂ to SO₃ also is neither upstream nor downstream; in most cases, it will be carried out in a separate and isolated reactor system that will not receive or process any organic compounds at all (although it may contain organic catalysts to speed up the SO₂ to SO₃ oxidation).

### Catalytic Surfaces, Zeolites, and Monoliths

Many chemical reactions involved in this invention use catalysts that are coated onto the surfaces of hard supporting materials, such as wire meshes, particulates in packed or fluidized beds, Zeolites or other porous solids, etc. “Supported” (or immobilized) catalysts are widely used in petroleum and chemical processing, since they allow expensive catalysts to be held and retained inside a reactor while large volumes of gas and/or liquid are pumped through the reactor. Accordingly, supported catalysts are well known, and are described in numerous books (such as Hayes et al 1997), articles (such as Raja et al 2000), and patents (various examples are cited below, most of which briefly mention one or more types of support and then focus on the catalysts that are coated onto the support surface).

This section contains a brief overview of three major types of catalysts, which are zeolite, SAPO, and monolith materials. This is followed by a discussion of how “ketene” compounds have been created by catalytic surfaces, which can help explain some of the principles that arise in creating a compound called “sulfene”, which is important in this invention.

“Zeolite” is the common name that has been given to porous “aluminosilicate” materials that contain silicon, aluminum, and oxygen, in crystalline lattices. The lattices have molecular-sized cavities (also called cages) that are connected to each other by smaller tunnels (channels), in repeating geometric formations. The sizes of the cavities can be modified, in controlled ways, by varying the formulation of a zeolite, thereby providing a certain zeolite formulation with cavities that are an optimal size to hold a particular type of molecule that will be processed by that zeolite. In addition, the narrow tunnels between cavities are small enough to force molecules being treated to line up in certain orientations, before they can pass from one cavity to the next, driven by pressure from a gas or liquid. In addition, the crystalline lattice that forms the cavities and tunnels can be embedded (or “doped”) with catalytic atoms, ions, or molecules. Because of these factors, zeolites and other porous catalysts can cause organic molecules to react in controllable ways that cannot be easily achieved by other materials.

As one example of how zeolite materials can be used, a major advance in methanol-to-gasoline (MTG) processing was discovered in the 1970's, when Clarence Chang and his coworkers at Mobil Oil Corporation (now Exxon-Mobil) discovered that if methanol is passed through certain types of Zeolite, methylene groups (—CH₂—) contributed by the methanol will begin condensing into chains, in a way that will create hydrocarbon liquids that can be used as gasoline. Early patents include U.S. Pat. Nos. 3,899,544 (Chang et al 1975), 4,076,761 (Chang et al 1978) and 4,138,442 (Chang et al 1979). Reviews include a book by Chang, Hydrocarbons From Methanol (Dekker, 1983), a chapter by Chang in Methanol Production and Use (W. Cheng & H. H. Kung, editors, Dekker, 1994), and Stöcker 1999, an extensive review article that cites 350 articles published by other authors. Stöcker 1999 is followed in the same journal by Keil 1999, which describes a number of commercial MTG facilities.

Zeolite materials that also contain phosphorus are often called “SAPO” materials, since they contain silicon, aluminum, phosphorus, and oxygen. During the research that followed the MTG discoveries of the early 1970's, it was discovered that processing of methanol on SAPO materials could create olefins, which are valuable unsaturated compounds that form the building blocks of plastics and polymers. “Methanol-to-olefin” (MTO) processing is described in U.S. Pat. No. 3,911,041 (Kaeding et al 1975) and in articles such as Liu et al 1999, Sassi et al 2002, and Dubois et al 2003.

However, because their cavities are connected by tunnels, “semi-permeable” or “meso-permeable” materials such as zeolite and SAPO tend to suffer from relatively large pressure drops, if gases or liquids are pumped through them. They also tend to suffer from fouling and clogging, due to the formation of sludge-like materials and/or “coke” (solid particulates or cakes, comparable to the materials used to make charcoal briquets or activated carbon powders). Therefore, zeolite or SAPO beds usually require periodic cleaning and regeneration, usually at high temperatures.

To minimize problems of clogging and pressure drops, many types of zeolites and similar catalytic materials have been developed that are designed to have surface activity only. Some of these materials have microscopic pores, comparable to pits, which encourage certain molecules to nestle into those pits in certain orientations, causing a certain atom or domain of the molecule to remain exposed to gases or liquids that are passed over the surface of the “charged” or “loaded” material. Other materials may be chemically treated, to bond positively- or negatively-charged atoms or groups to their surfaces, usually at a controlled density or spacing. Since these types of surface-only materials do not need to have a gas or liquid actually pass through flow channels in the material, they often are used in particulate forms, such as in packed or fluidized beds.

Alternately, a different class of catalytic supports has been developed with porous materials that have tiny flow-through channels that are essentially straight and linear, passing through the otherwise solid material. These materials, called “monoliths”, are commonly manufactured in the form of round discs (often called “cakes”) that will fit into cylindrical containers, which usually are provided with inlet and outlet filters to hold the monolith in place while a gas or liquid flows through it. Because the flow channels in monoliths are essentially straight, linear, and parallel, and do not have any constrictions or changes in internal size, monoliths can provide higher and faster flow rates, lower pressure drops, and fewer clogging and fouling problems, compared to zeolites or other materials having non-linear flow channels. Therefore, catalytic monoliths are often used in devices that cannot be easily or periodically shut down and cleaned out (such as catalytic converters that remove pollutant gases from automobile exhausts).

Monoliths can be prepared with various channel sizes and densities, usually expressed as channels per square inch (cpsi). Monoliths that handle gases usually have cpsi values ranging from about 400 to over 1000. Monoliths that handle liquids or foams require larger channels, with correspondingly lower cpsi numbers, to achieve an optimal balance between (i) reasonable and acceptably low “pressure drops” across the material, and (ii) desirably high levels of contact between the solid surfaces and the liquids flowing through the channels. Because thousands of flow channels pass through a monolith of any substantial size, very large total surface areas, inside the thousands of tiny flow channels, will be contacted by a gas or liquid that passes through a monolith.

The material that provides the porous supporting structure of a catalytic monolith is usually called the “support”. It is sometimes referred to as the “substrate”, to distinguish it from the catalytic coating material, but that term is confusing and should be avoided, since “substrate” also refers to a chemical compound that is acted upon by a catalyst, enzyme, or other chemical reagent.

In general, monolith supports must be able to withstand strong acids (which eliminates most metals and alloys) and high temperatures (which eliminates most plastics and starch-type polymers). These requirements usually lead to the use of minerals and/or ceramics, which frequently contain silicon and oxygen (often referred to as “silicate” materials), which are comparable to quartz but with porous lattices that provide flow channels. Support compounds such as cordierite, mullite, or silicon carbide are widely used, and are sold by companies such as Corning Inc. (www.corning.com) and Rauschert Process Technologies (www.rauschertus.com).

Various types of surfaces can be provided on a hard support material. One class of such surfaces is called “porous” supports, which implies irregular and uneven surfaces, which may be created by the material itself, or by surface treatment processes such as anodic or acidic etching. Alternately, “abraded” supports usually provide smoother surfaces, formed by processes comparable to sanding. The suitability of any such porous, abraded, or other support surface, for hydrocarbon or chemical processing as disclosed herein, can be evaluated through routine testing.

All of the foregoing relates to the support material, which in most cases is presumed to be chemically inert, and uninvolved in the molecular rearrangements involved in a chemical reaction. Regardless of how an inert supporting material is prepared, or what physical form it is in, the real value and functionality of any supported catalyst will depend on how the surfaces are “activated” (also called “functionalized”). This usually is done in either of two manners. In one approach, the surface of a hard and presumably inert support material is coated with catalytic atoms, ions, or groups; the term “coating” is used broadly herein, and involves plating, liquid immersion, sputter coating or other gaseous diffusion, or any other process that creates a surface layer that is somehow different from an underlying support material). The second approach involves incorporating the catalytic atoms, ions, or groups into the reagents used to form a supported catalyst, in some manner that distributes the catalytic atoms, ions, or groups throughout the resulting material. This second method is conventionally used to make zeolite, SAPO, and monolith materials that require catalytic “dopant” atoms to be positioned at regular intervals in a crystalline lattice. Either of those two approaches can be regarded as “functionalizing” a catalyst, in a way that turns an inert supporting material into a chemically active material that can help trigger, drive, and control valuable reactions.

This is a brief and simplified overview, intended to help readers who do not specialize in these types of materials develop a basic understanding of how these types of materials are made and used. Any reader who wants more information on supported catalysts can locate numerous articles and books on the subject, and in websites that provide lecture and course notes for chemistry courses at various universities.

Chemists and researchers who specialize in working with zeolites and similar materials have formed an organization called the International Zeolite Association, which runs a highly useful website, www.iza-online.org. The IZA has been designated by the IUPAC (the International Union of Pure and Applied Chemists) as having official responsibility for nomenclature and other matters relating to zeolites and other compounds with structural lattices that enable them to function as molecular sieves and/or catalysts. The IZA has three major working groups, devoted to structures, synthesis, and catalysis. Anyone who wishes to identify zeolite or similar compounds that offer good candidates for processing a particular chemical (such as methanesulfonic acid, one of the key intermediates in this invention) can quickly locate experts in the field, through the IZA. In addition, any company that sells zeolite compounds for chemical processing, either as a manufacturer or distributor, will have on its staff at least one technical expert who can advise potential purchasers on: (i) which compounds offer the best candidates for early evaluation, for carrying out a particular reaction; and, (ii) the names of experts who can be consulted for more information.

At least one professional journal, Microporous and Mesoporous Materials, is entirely devoted to zeolite and similar materials, and several other journals (including the Journal of Molecular Catalysis, the Journal of Physical Chemistry, and Fuel Processing Technology) frequently publish articles on processing and research using porous catalysts. Accordingly, experts who specialize in particular formulations, or in processing certain classes of chemicals on porous catalysts, can be located by contacting an editor who works with one of those journals, or by reviewing the titles of articles that have been published in such journals.

Also, methods and machines have been developed for screening large numbers of candidate catalyst formulations, in a rapid and automated manner. These methods and machines are described in articles such as Muller et al 2003, and other articles cited therein. Such devices use, for example: (i) reactors with multiple parallel tubes, each tube containing a different candidate catalyst, or (ii) titer plates with multiple wells, each well containing a candidate catalyst. When a certain reagent is passed through or loaded into all of the tubes or wells, the product generated by each individual tube or well (and therefore by each candidate catalyst) is collected separately, and delivered to an automated analytical device, such as a mass spectrometer or chromatograph. The tubes or wells that created the highest yields of the desired compound can be identified, and the exact content of the catalysts in any tubes or wells that resulted in good and desirable yields can be identified and studied more closely. For example, the best-performing candidate catalyst from one round of tests can be used as a “baseline” or “centerpoint” material, in a subsequent round of tests that will use variants that resemble the best-performing catalyst from the previous round of screening. Those variants can include known and controlled compounds, having exact compositions; alternately or additionally, “combinatorial chemistry” methods and reagents can be used, to generate random or semi-random variants of a material that provided good results in an earlier screening test. Accordingly, these types of automated screening systems offer powerful and useful tools for rapidly identifying and/or improving porous catalyst formulations that can efficiently promote any particular desired reaction.

Another factor worth noting involves catalytic agents that use symphoric, anchimeric, or “neighboring group” effects to enable “two-handed” manipulation MSA or other compounds. MSA has two very different domains, methyl and sulfonic. In the pathway shown in FIG. 3 (discussed below), the silicate support merely uses hydroxy groups to attract and interact with MSA. More potent and efficient catalysts might be developed, by providing a catalytic surface with two different types of functional agents, allowing one type of catalytic group to attract and interact with the sulfonic portion of MSA, while the second type of catalytic group attracts and interacts with the methyl portion. This factor can be better understood, if the reader will consider additional comments in PCT application PCT/US03/35396 (published in May 2004), about the symphoric and/or anchimeric traits of a bromate-sulfate reagent that can convert methane into a methyl-bis-sulfate compound.

### Surface Adsorption, Transition States, and Ketene Chemistry

Once a reader has a working knowledge of how surface-active catalysts can be formulated, and how various different classes of physical configurations are known and available, the next step is to consider how gases or liquids will interact with those types of catalytic materials that are supported on monolithic, particulate, or other solid supports. Several terms and concepts are briefly explained below, then these terms are considered in the context of a prior art method for making a compound called ketene, which is analogous in some respects to sulfene, a key intermediate in this invention.

The term “adsorbed” refers to a molecule that is closely associated with the surface of a solid material. In the types of reactions of interest herein, this “close association” will last only very brief time (typically measured in milliseconds), and it will occur solely during the transition from one state to a different state (especially in reactions that run at high temperatures). This association usually is initiated by some form of charged (positive-to-negative) attraction, in which hydrogen protons or other positively-charged ions or atoms are attracted to localized negative charges (such as unshared electron pairs, on the surfaces of exposed oxygen atoms). This charge attraction draws a liquid or gaseous compound into close proximity with certain molecules on the surface of the support material. When the “lowest energy” distance is reached, the association between the reagent and the catalytic coating then progresses through one or more types or stages of “coordinate” or “metastable” bond transitions, which may or may not rise to the strength of covalent bonding.

Because of various factors, it is not always possible to clearly and definitely categorize the various transitional molecules and bonds. For example, an atom or molecule can pass through transitional states that can be regarded as intermediate or halfway points, somewhere between an ionic state, and a radical state; similarly, the types of “coordinate” or “metastable” bonds that arise can be at an intermediate or halfway point between an ionic attraction or a covalent bond. Therefore, in any narrative descriptions herein (in Barteau's pathway for the production of ketenes, and in the Applicant's pathway for the production of sulfene), any reference to any transitional or intermediate state of any particular atom, ion, or radical can be regarded as merely a convenient reference term, comparable to an estimate or approximation, intended to provide a map or narrative description of the relevant terrain, for the use of experts who wish to analyze these types of reactions in greater detail. Much more information on intermediate states, transitional bondings, and “hybridized” valence-shell structures that can occur during the course of catalytic reactions, is available in numerous published reference works (such as, for example, March's Advanced Organic Chemistry, by M. Smith and J. March, Wylie Interscience, 2001).

It should also be kept in mind that the patent law generally does not require a theory, to explain a new invention. Instead, the patent law requires a description of a feasible method for accomplishing a desired result, in adequate detail to teach those skilled in the art how to mix the reagents, and run the process. In this invention, the result and the invention center on the conversion of MSA into sulfene, and the subsequent conversion of sulfene into other useful chemicals, such as olefins. Accordingly, any specific or postulated reaction steps, and any transitional or intermediate bondings or molecular complexes that are hypothesized, proposed, or otherwise discussed herein, are not essential to carrying out this invention. Instead, this invention resides in the recognition and disclosure of several practical and useful results (including but not limited to the realization that MSA, which can be formed from “waste” methane, can be pushed into forming a sulfene intermediate, and the sulfene will then react in ways that will form ethene or other valuable compounds). Those and other practical teachings herein form the essence of this invention, and any discussion of postulated, hypothesized, probable, or modeled atomic or molecular interactions or transition states is offered merely as additional commentary, in the hope that such commentary might be useful to experts who wish to study and analyze these or similar reactions in greater detail. This commentary is not asserted to be the final answer or definitive theory, and instead should be regarded as suggesting certain places and references where experts might wish to look, among the huge mass of published and available material.

The concepts above, regarding liquid or gaseous chemicals that are temporarily “adsorbed” onto the activated surfaces of catalysts, in ways that: (i) promote or enable a desired reaction, and then (ii) release the product compound from the catalytic surface, can be illustrated by a discussion of “ketene” compounds, described by Mark Barteau in articles such as Barteau 1996. The simplest ketene compound is H₂C═C═O. More complex ketenes also can be created, by replacing either or both of the two hydrogen atoms with other groups, to create R₁R₂C═C═O, where R₁ and R₂ are variables.

In a series of reactions described in Barteau 1996, the suffix (ad) was used to indicate a compound that was temporarily “adsorbed” to the surface of a solid catalytic material. The suffix (g) referred to a compound that was released, from the catalytic surface, as a gas (in the case of water, this will be steam, since these reactions are carried out at high temperatures). The suffix “O(/)” referred to “surface oxide anions” (i.e., oxygen atoms or ions that are closely associated with, but not covalently bonded to, the support surface).

One reaction described in Barteau 1996 involved the processing of acetic acid, on silicate supports using catalytic surfaces that contain exposed oxygen atoms, to form ketene. In the first step of Barteau's pathway, written as:

CH₃COOH+O(/)-->CH₃COO(ad)+OH(ad)

a molecule of acetic acid ionizes, and transfers its hydrogen proton to an oxygen atom/ion on the silicate surface. This causes the acetate ion (which has a negative charge, after losing its hydrogen proton) to be attracted to, and adsorbed onto, the support surface, which has positive charges on it due to hydrogen protons it is receiving from the acetic acid solution.

In the second step of Barteau's pathway:

CH₃COOH+OH(ad)-->CH₃COO(ad)+H₂O(g)

a second molecule of acetic acid ionizes, and transfers its hydrogen proton to an adsorbed hydroxy group on the support surface. This causes the hydroxy group to be converted into a full molecule of water, which leaves the support surface, in the form of steam. The second acetate anion becomes adsorbed onto the support, which continues to have positive charges on it due to other hydrogen protons that are being donated to it by the acetic acid solution that continues to contact the support.

In the third step of Barteau's pathway:

CH₃COO(ad)-->H₂C═C═O(g)═H(ad)+O(/)

an acetate ion that was adsorbed onto the support surface rearranges its molecular configuration. A hydrogen atom (or proton) on the methyl group leaves, and becomes adsorbed on the support surface. The electron (or electron pair) that formed an H—C bond, in the acetate ion, moves around that carbon atom, and forms a double bond between the methyl carbon, and the other (carboxy) carbon. This electron shift also causes the single-bonded oxygen ion from the second (carboxy) carbon to leave the acetate ion; it will become adsorbed on the support surface, thereby regenerating that support as a catalyst, rather than as a consumed reagent.

The fourth step of Barteau's pathway:

H(ad)+OH(ad)-->H₂O(g)

partially balances out the equations above, by releasing water, as a gas (steam). This water is formed from the hydrogen atom (or ion) that was adsorbed on the support surface in the third reaction step, and the hydroxy group (or ion) that was adsorbed on the support surface in the first reaction step.

It should be noted that the reactions listed above are only partially balanced; an acetate ion remains adsorbed on the support, presumably able to rearrange itself to form additional ketene while also releasing H and O for adsorption on the support. As noted by Barteau in the text following the reactions listed on page 1423 of his article, side reactions also occur, including unselective decarboxylation of the adsorbed acetate ions. This releases CO₂, and it also deposits hydrocarbon fragments (from the methyl groups) on the support.

After studying and analyzing a number of articles, books, and patents (including the 1996 article and several other articles and patents by Barteau, and by other authors and inventors), the Applicant herein suspected that similar processes may also be able to occur if MSA (rather than acetic acid) is processed on a comparable type of support. To inquire into that possibility, he had the MSA reaction analyzed by computer modeling, with the paid assistance of a doctoral candidate who had access to powerful computers at a university. This modeling used the Amsterdam Density Functional software, release 2.3.3, sold by Scientific Computation and Modeling (www.scm.com), described in articles such as te Velde et al 2001. The results were positive and promising, and are described below, in the Detailed Description section.

Accordingly, the remainder of this application describes and claims: (i) various enhancements of the MSA pathway, that were identified or developed by the Applicant since the filing of the earlier PCT applications. These options and enhancements include:

(1) processing options that can be carried out, either by using the MSA-to-methanol system as a starting point, or by branching out from that pathway at one or more junction points (such as by converting MSA into an unstable anhydride called sulfene, H₂C═SO₂, which can then be processed in various ways to create stable and valuable products, such as liquid hydrocarbon condensates that can be used as fuels or feedstocks;

(2) enhanced methods for oxidizing SO₂ into SO₃, so that the cycle of sulfur compounds, shown on the right side of FIG. 2, can be carried out more efficiently and economically; and,

(3) methods and catalysts for carrying out a similar and related type of reaction, which will cause methyl radicals to react with carbon dioxide, rather than SO₃, in a way that will form acetic acid (a useful and valuable chemical) rather than MSA, in a way that can reduce carbon dioxide emissions into the atmosphere by exhaust gases from power plants, factories, and other sources.

One object of this invention is to disclose various enhancements and options that can be used to expand and improve upon various teachings of two previous PCT applications (serial numbers PCT/US03/035396, published as WO 2004/041399, and PCT/US2004/019977) that were previously filed by the same Applicant.

Another object of this invention is to disclose various processing pathways and options, using methane-sulfonic acid (from methane gas) as a feedstock or intermediate, to create various types of valuable organic chemicals.

Another object of this invention is to disclose processing pathways and options, using methane-sulfonic acid (from methane gas) to create an unstable anhydride intermediate called sulfene (H₂C═SO₂), which can be processed to create stable and valuable organic chemicals.

Another object of this invention is to disclose enhanced methods for oxidizing SO₂ into SO₃, so that the cycling of sulfur compounds, as part of a larger processing system that converts methane into methanol or other compounds, can be carried out more efficiently and economically.

Another object of this invention is to disclose methods and catalysts for causing methyl radicals to react with carbon dioxide, to form acetic acid, thereby forming a valuable chemical while also reducing carbon dioxide emissions into the atmosphere.

These and other objects of the invention will become more apparent through the following summary, drawings, and detailed description.

### SUMMARY OF THE INVENTION

Enhancements and options are disclosed for chemical processing methods described previously by the Applicant, for converting methane into methanol or other organic compounds, via methyl radicals and methane-sulfonic acid (MSA). A major set of options and enhancements, which are the primary focus of the claims herein, relate to converting MSA into an unstable and highly reactive anhydride intermediate called sulfene, H₂C═SO₂. This compound is a potent and useful donor of methylene groups (—CH₂—), which can be used for purposes such as creating heavier liquid fuels or olefin compounds, or for creating plastic or polymeric compounds in particulate or other form. Methods and catalysts for working with sulfene are disclosed, such as the use of tungsten or other metal catalysts that have been driven to a +6 oxidation state, for efficiently converting sulfene into ethylene, in a “single pot” conversion that uses MSA as the feedstock, which makes optimal use of the sulfene intermediate.

Other options and enhancements disclosed herein can be divided into several categories, including: (1) “upstream” processing options, such as improved methods for making Marshall's acid, one type of radical initiator that can be used to convert methane into MSA; (2) the use of a dimethyl variant of Marshall's acid, created by electrolysis of MSA, as a radial initiator that is easier to handle and that will not create any unwanted byproducts; (3) various “downstream” processing options for treating MSA, such as methods for treating methyl-ester impurities that may be created during MSA cracking or other processes.

### BRIEF DESCRIPTION OF THE DRAWINGS

FIG. 1 depicts several known chemical reactions that can “activate” methane (CH₄) by removing a hydrogen atom (both a proton and an electron), to convert the methane into a methyl radical (H₃C*, where the asterisk represents an unpaired electron).

FIG. 2 depicts a reaction system that combines methyl radicals (H₃C*) and sulfur trioxide, to form methane-sulfonic acid (MSA) by a multi-step process that creates a new methyl radical. This establishes a chain reaction, and the newly-created methyl radicals will react with newly-added SO₃. MSA can be removed from the vessel and sold as a product, used as a reagent, or “cracked” to release methanol (which can be shipped as a liquid, or used as a feedstock for other reactions) and sulfur dioxide (which can be oxidized to SO₃ and recycled back into the reactor).

FIG. 3 depicts transitional intermediates that are likely to be formed if MSA is dewatered with the assistance of a silicate monolith material having hydroxy groups on its surface.

FIG. 4 depicts a reaction of two molecules of sulfene (H₂C═SO₂) to form ethene, in gaseous form. This reaction releases gaseous SO₂, which can be oxidized to SO₃ and recycled back into the reactor vessel that is used to convert methane into MSA.

FIG. 5 depicts an alternate candidate pathway for dewatering MSA to form sulfene, using tungsto-phosphoric acid (also called phospho-tungstic acid).

FIG. 6 depicts an alternate candidate pathway for making sulfene, using a methyl-MSA compound that is reacted with methanol, which is recovered and recycled.

FIG. 7 depicts a pathway for using sulfene to convert ethene into cyclopropane, which can be converted into propene (propylene), propanol (propyl alcohol), or other products.

FIG. 8 depicts a candidate pathway for converting sulfene into ethylene, using a solid-supported tungsten or other metal catalyst that has been driven to a +6 oxidation state.

FIG. 9 depicts a reaction pathway that proceeds through an “outer” anhydride form of MSA, formed by condensing two molecules of MSA while removing a water molecule.

FIG. 10 depicts a potential polymerization pathway, in which sulfene will insert multiple methylene groups into a growing alkane molecule or derivative, which may be a branched alkane or derivative if certain types of diimine or other catalysts are used.

FIG. 11 shows a pathway that enables a vanadium diformate catalyst to convert SO₂ into SO₃, using pathways that appear from computer modeling to be thermodynamically favorable.

FIG. 12 is a schematic depiction of a system for converting SO₂ to SO₃, which uses heat from the SO₂ oxidation reaction to heat MSA from its relatively cool formation temperature, up to a much higher cracking temperature.

FIG. 13 depicts electrolytic formation of a dimethyl variant of Marshall's acid, referred to as di(methyl-sulfonyl) peroxide (abbreviated as DMSP), and the use of DMSP as an initiator that will not create any unwanted byproducts when used to bond methane to SO₃ to form MSA.

FIG. 14 depicts a candidate pathway for converting MSA into a fuel called dimethyl ether.

### DETAILED DESCRIPTION

As summarized above, this application focuses largely on the manufacture and use of sulfene, H₂C═SO₂, a highly reactive anhydride form of MSA that can be used to manufacture olefins, liquid fuels, or other valuable compounds from stranded methane gas. These disclosures are addressed in the immediately following subsections.

After that discussion of sulfene, several additional disclosures are provided, including:

(1) options and enhancements in “upstream” processing (i.e., reagents, devices, and methods used to manufacture MSA as an intermediate), including the use of a dimethyl variant of Marshall's acid as a radical initiator that is easier to handle, and that will not create any unwanted byproducts; and,

(2) various “downstream” processing options for treating MSA, such as methods for treating methyl-ester impurities that may be created during MSA cracking or other processes.

These disclosures are included herein, for two reasons. First, they are believed to be necessary to ensure disclosure of the “best mode” for carrying out the overall processing system described herein.

The second reason is this: these disclosures are believed to reveal substantially improved ways of making good, efficient, humanitarian, and benevolent use of energy resources, while providing better methods of environmental protection as well. Accordingly, rather than generating a profusion of dozens of confusingly different-yet-interrelated patent applications, all of which would need to be located and studied carefully, the energy-conserving and environment-protecting goals of this technology (and of the Applicant) can be better served by compiling a number of discoveries and advancements into a smaller number of patent applications. By taking that approach, a single inventor can help establish a functional and efficient foundation and framework that can help support and enable a clear understanding, by all interested parties, of what is being taught, and how it can be developed as rapidly as possible into commercial and industrial use that will help the public, and the planet.

Accordingly, the immediately following subsections focus on making, and then using, sulfene. After those subsections, various additional subheadings are provided.

### Synthesis of Sulfene

As briefly summarized above, methods are disclosed herein for converting methane-sulfonic acid (MSA, which can be prepared from methane gas as described in PCT applications PCT/US03/035396 and PCT/US04/019977) into an “inner anhydride” called sulfene, H₂C═SO₂ (also called thioformaldehyde dioxide).

Sulfene is highly unstable. If formed in large quantities and/or high concentrations, two molecules of sulfene can react with each other, in a rapid and highly exothermic reaction, to form ethylene (also called ethene), H₂C═CH₂, a valuable olefin used in the manufacture of plastics and polymers. Since ethylene is a valuable product, additional disclosures are provided below on catalysts that can help promote that reaction, to cause it to produce ethylene with greater yields, selectivity, and purity.

Under other conditions, sulfene can be used as a “methylene transfer agent”, which can insert methylene groups (which can be represented as —CH₂— or as H₂C:) into other compounds, as discussed in more detail below. This reaction can be used to convert various hydrocarbon compounds (include gaseous or other relatively light or “thin” hydrocarbons, such as short-chain hydrocarbons with 2 to 5 carbon atoms) into larger and heavier compounds, which generally will be easier to handle (since they will be less volatile) and more valuable (since they will have higher energy density). As just one example, if sulfene reacts with ethylene, the methylene group from sulfene will convert the ethylene into cyclopropane, which can be (1) used as a chemical feedstock, which will be highly reactive due to its stressed bond angles, (2) isomerized to form propylene (also called propene), another valuable olefin, or (3) hydrated to form propyl alcohol, a valuable chemical and a gasoline additive or substitute. In general, transfer of methylene groups into most types of gaseous and/or volatile hydrocarbons will decrease their volatility, making them easier to store, transport, and handle, and will also increase their energy density, utility, and value.

Regardless of whether sulfene reacts with itself to form ethylene, or acts as a methylene transfer agent that adds a CH₂ group to a hydrocarbon molecule, the SO₂ group in sulfene typically will act as a leaving group, causing most sulfene reactions to release SO₂ in gaseous form. This gas can be collected, oxidized back into SO₃, and returned to the reactor vessel that is being used to convert methane into MSA, in a recycling operation that minimizes wastes and unwanted byproducts.

The conversion of MSA into sulfene requires the “dewatering” of MSA (H₃CSO₃H). This “dewatering” process can also be called dehydration; however, dehydration is a broader and less precise term, and can be used whenever hydroxy groups (HO—) are removed. Accordingly, the term “dewatering” is preferred herein, to indicate that two hydrogen atoms and an oxygen atom must be removed from each molecule of MSA that is fully converted into sulfene. The term “anhydride” can also be used, to refer to molecules of MSA from which water molecules have been removed. If desired, terms such as “inner” or “internal” dewatering or dehydration can be used, to indicate that a complete molecular equivalent of water has been or will be removed from a single molecule of MSA, to form sulfene; however, it should be understood that in most cases, each water molecule released by MSA during a dewatering process typically will contain a hydroxy group from the sulfate domain of one MSA molecule, and a hydrogen proton from the methyl domain of a different molecule of MSA.

There also is an “outer” anhydride of MSA, H₃CSO₂—O—SO₂CH₃, formed by condensing two molecules of MSA while removing a single molecule of water. Under some conditions, this intermediate can rearrange to form sulfene, while releasing MSA. Outer anhydrides of MSA can be useful, and are discussed below.

Several candidate methods for converting MSA into sulfene are disclosed herein. Preferred methods for different manufacturing sites may depend on various factors, such as flow rates and flow rate consistency levels at that site, the purity levels and contaminant loads in the methane stream as well as the MSA intermediate, the ability of other equipment at a site to handle any wastes or unwanted byproducts that may be created by the various candidate methods, and the targeted purity levels for sulfene or downstream products that will enable operations at a particular site to be optimized on an economic basis. Accordingly, any candidate method disclosed herein (and any other candidate method that is currently known or hereafter discovered) can be evaluated, both in batch-processing and continuous-flow modes of operation, to determine its suitability and economics for use at any particular site.

It should be recognized that until this point in time, sulfene has received little attention from chemical researchers, mainly because of two reasons: (i) it is unstable, and will not last long even when created; and, (ii) the only prior art methods for preparing it are difficult and tedious, and generate too much toxic and hazardous waste to enable sulfene manufacture to be used as a practical and economic route toward creating other valuable products. Two of the relatively few items published to date on sulfenes are: (i) chapter 17, by King and Rathore, in a book edited by Patai and Rappaport 1991, and (ii) Prajapati et al 1993, which describes a method of generating sulfene that consumes SOCl₂ and generates hydrochloric acid as a byproduct. To the best of the Applicant's knowledge and belief, neither of those items, both published more than a decade ago, ever led to any significant commercial or industrial activity using sulfene.

However, the level of interest in sulfenes may increase dramatically, after the disclosures herein have been made known to chemical and petroleum companies and academic researchers, since a feedstock that can be used to make sulfene (i.e., MSA from stranded methane gas) is likely to become available soon, at lower cost and greater quantities than any currently-available supplies of MSA.

Accordingly, the disclosure of synthesis pathways that pass from stranded methane through MSA and then through sulfene, to create olefins or other valuable compounds, is likely to trigger substantial research interest, and it is likely that these pathways can be supplemented and enhanced by other methods. Accordingly, these pathways are being disclosed at an early stage of evaluation, after they have been tentatively confirmed in batch processing but before they have been tested or evaluated in continuous-flow operations, to confirm that these pathways are indeed feasible, and to help researchers and engineers interested in this field of research become familiar with various factors and options.

Several of the synthesis pathways that pass through sulfene will require: (i) elevated temperatures, to overcome certain thermodynamic barriers, and (ii) active removal of water (usually in the form of steam), to pull certain reactions in the desired direction. In some pathways, water can be removed from MSA directly, as discussed relative to hydrated silica, below. Alternately, in some pathways (such as pathways involving methyl-methanesulfonate ester, or the outer anhydride of MSA), water may be removed from certain components prior to the creation of sulfene. This can provide benefits in various types of downstream processing.

Three main categories of candidate pathways are described below, for synthesizing sulfene. Each candidate pathway is discussed under its own subsection.

### Candidate Pathway #1: Solid-Supported Catalysts

The first candidate pathway disclosed herein for dewatering MSA to form sulfene uses catalytic materials on the surfaces of solid supports. Catalysts that are coated onto (or otherwise made accessible on) the surfaces of solid support materials are widely used in the petroleum and chemical industries, because they allow expensive catalytic materials to be retained inside a reactor while large volumes of gas or liquid are pumped through the reactor. As discussed in the Background section, the types of solid-support catalysts disclosed herein can be provided in any of several candidate forms, such as: (i) “monolith” materials, with essentially linear and parallel flow channels passing through a porous material that can be manufactured in a “cake” or similar form that can be secured inside a pipe or reactor; or (ii) particulate materials, which can be loaded into a packed bed, fluidized bed, stirred reactor, or comparable device.

The conversion of MSA into sulfene will be comparable in some respects to the conversion of acetic acid into ketene (H₂C═C═O), which is summarized in the Background section by describing certain reactions disclosed in Barteau 1996. After studying and analyzing a number of published articles by Barteau, and other articles and patents by other authors and inventors, the Applicant herein suspected that similar processes may occur if MSA (rather than acetic acid) is processed on a suitable supported catalyst.

To evaluate that possibility, he had the MSA reaction analyzed by computer modeling, using the Amsterdam Density Functional software, release 2.3.3, sold by Scientific Computation and Modeling (www.scm.com), described in articles such as te Velde et al 2001.

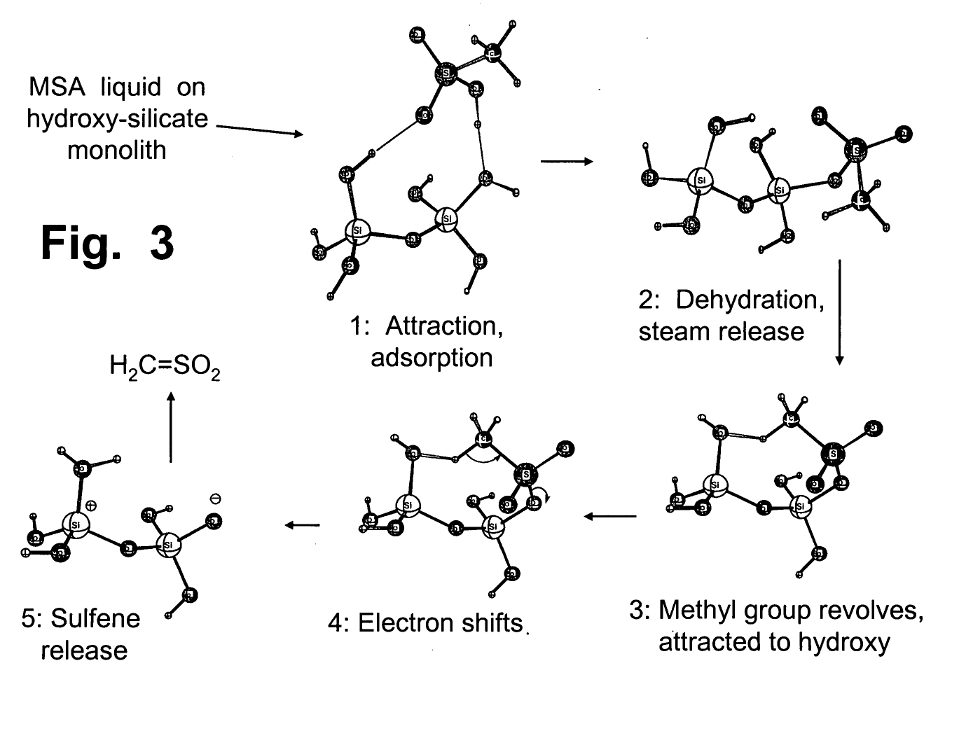

Results from that computer modeling are provided in FIG. 3, which depicts a three-dimensional model of how a molecule of MSA can interact with a silicate support that has hydroxy groups exposed on its surface, represented by (Si(OH)₃)₂O.

In the initial step of this series of reactions, one of the double-bonded oxygens of the sulfate portion of MSA is attracted to one of the hydrogen protons on the silicate support, and the hydrogen proton on the hydroxy group of the sulfate portion of MSA is attracted to one of the oxygen atoms in one of the hydroxy groups on the silicate support. At the same time, a coordinate bond between the two silicon atoms is also disrupted or reconfigured. This combination of steps forms an adsorbate, in which MSA has become closely associated with the silicate surface. This attraction and affiliation is an exothermic reaction that occurs spontaneously, with a ▴E value of −10.72 kcal/mol (kilocalories per mole). The term ▴E refers to bonding energies, which correspond to ▴H (enthalpy) values when certain “zero point energy” (ZPE) corrections are made, as known to those skilled in the art.

In the second step, which will occur only at elevated temperatures, a water molecule will leave, most likely containing a hydroxy group that initially was on the silicate support, along with a hydrogen proton or atom from the sulfate group of the MSA. This leads to formation of a silicon-oxygen-sulfur linkage, on the right side of the molecule shown in FIG. 3. This reaction is endothermic, which means it requires energy input, with a calculated value of ▴E=10.79 kcal/mole. Since that figure is nearly equal to the exothermic energy release of the initial step, the net result is nearly “thermoneutral”, and it will occur on an equilibrium basis, at high temperatures. This equilibrium can be shifted and pulled in the desired direction, to provide greater yields, by actively removing from the reactor vessel any water that is released (in the form of steam) from that reaction.

Accordingly, the first and second steps, taken together as described above and illustrated in FIG. 3, can be written as follows:

H₃C—SO₃H+OH(ad)-->CH₃SO₃(ad)+H₂O

In the next step of the reaction, the MSA residue will pivot and swing around an “axle” that is provided by the silicon-oxygen-sulfur linkage, until one of the positively-charged hydrogen protons, on the methyl group of the MSA residue, approaches a negatively-charged hydroxy group, which might be bonded to the same silicon atom that the sulfur is bonded to, but which more likely will be bonded to some other nearby silicon atom in the matrix of the silicate support.

In the fourth step, three proton and electron shifts take place, which function together to set the stage for the disengagement of sulfene from the substrate. In one shift, the hydrogen proton that formed the bridge between the methyl group of MSA, and the hydroxy group of the substrate, shifts toward the hydroxy group of the substrate, thereby weakening its bond and its attraction to the carbon atom of the MSA. In a second coordinated shift, the electron pair that previously formed the carbon-hydrogen bond (which has now become weakened because of the hydrogen proton's attraction to the hydroxy group on the substrate) will be pulled toward the electronegative sulfur atom. This sets up the formation of a double bond between the carbon atom and the sulfur atom. In the third coordinated shift, this formation of the double bond between the sulfur and the carbon will weaken the single bond between the sulfur atom, and the oxygen atom that forms the sulfur-oxygen-silicon linkage.

After these three shifts occur, in what can be regarded as the fourth step of the reaction, the fifth step can occur, in which the MSA residue will detach from the silicate support, in a way that creates a double bond between the carbon and the sulfur. The hydrogen proton from the methyl group, and the oxygen atom from the sulfate group, will both be left behind, adsorbed on the solid support material. When this detachment occurs, the H₂C═SO₂ molecule that remains from the original MSA has become sulfene.

The detachment of the sulfene from the solid support will leave a polarized condition on the support, with a positive charge on the hydroxy group that received a hydrogen proton from the methyl group of MSA, and a negative charge on the oxygen atom that was donated by the sulfate group of MSA. Under the acidic conditions that will exist in the system (due to the continued addition of fresh MSA, an acid, to the system), those localized charges can be resolved in any of numerous ways involving migrating or mobilized protons, in ways that will regenerate the hydroxy groups on the support, and restore the silicate support, thereby rendering it a catalyst, rather than a consumed reagent.

Accordingly, when the third, fourth, and fifth steps in the pathway are combined together and written in the aggregate, they become:

CH₃SO₃(ad)-->CH₂SO₂+OH(ad)

When this reaction is combined with the reaction above, and then balanced by eliminating identical items on the left and right sides, the net result of both reactions becomes:
ti CH₃SO₃H-->CH₂SO₂+H₂O(released as gas/steam)

Table 1 provides the ΔH (change in enthalpy) values and the ΔG (change in Gibbs free energy) values for the formation of ethylene via either of two routes: (1) from acetic acid via ketene, as described in Barteau 1996, for comparative purposes; and, (2) from MSA via sulfene, as disclosed herein. These units are in kilocalories per mole, but since two moles of sulfene make only one mole of ethylene, attention needs to be paid to whether the one-mole compound is on the left or the right side of the reaction arrow, as discussed in more detail following the table.

Both sets of values were calculated at 3 different temperatures, which are 300, 600, and 900 Kelvin. The Kelvin scale begins at theoretical absolute zero, at which all atomic motion completely stops. A Kelvin temperature can be converted into centigrade by subtracting 273.15; therefore, the modeling temperatures were equal to 26.85, 326.85, and 626.85° C. Those temperatures are equal to 80, 620, and 1130°, in the Fahrenheit scale, which is mentioned to emphasize the range they cover, and to point out that the lowest modeled temperature (which is close to room temperature) would not be useful or practical for manufacturing ethylene from MSA, since the initial barrier to reach sulfene is too high.

TABLE 1
THERMODYNAMICS OF KETENE OR SULFENE REACTIONS

| Ketene reactions | ΔH | ΔG-300K | ΔG-600K | ΔG-900K |
|---|---|---|---|---|
| CH₃COOH -> H₂C═C═O + H₂O | 30.66 | 18.36 | 4.5 | −8.82 |
| 2H₂C═C═O -> C₂H₄ + 2CO | 5.61 | −3.63 | −12.08 | −19.87 |
| Sulfene reactions | | | | |
| CH₃SO₃H -> H₂C═SO₂ + H₂O | 34.83 | 23.30 | 11.79 | 1.12 |
| 2CH₃SO₃H -> 2H₂C═SO₂ + 2H₂O | 69.66 | | | |
| 2H₂C═SO₂ -> C₂H₄ + 2SO₂ | −54.09 | −66.67 | −77.68 | −87.85 |
| SO₂ + ½O₂ -> SO₃ | −20.86 | | | |
| Overall reaction: | −26.15 | | | |
| 2CH₃SO₃H + O₂ -> C₂H₄ + 2H₂O + 2SO₃ | | | | |

The conversion of MSA to sulfene will require a very hot reactor. One candidate class of reactors that can handle such temperatures includes reactors that contain monolith supports, made of quartz-like but porous silicate materials having essentially linear and parallel flow channels, as described in the Background section.

It should also be noted that many scientific articles refer to “adiabatic” monolithic reactors. The term “adiabatic” indicates that a reaction is carried out without adding external heat to the reactor, and without using heat exchangers or other means to actively draw heat away from the reactor vessel. When used during research, that approach can simplify and clarify various data and calculations. However, when highly exothermic (heat-generating and heat-releasing) reactions are carried out on a commercial scale, in large manufacturing operations, it would be wasteful to let the heat simply dissipate, rather than putting it to productive use. Therefore, it is presumed that in most industrial operations, any reactor that generates intense heat from an exothermic reaction will be surrounded by some type of heat exchanger, and will not be run as an adiabatic process.

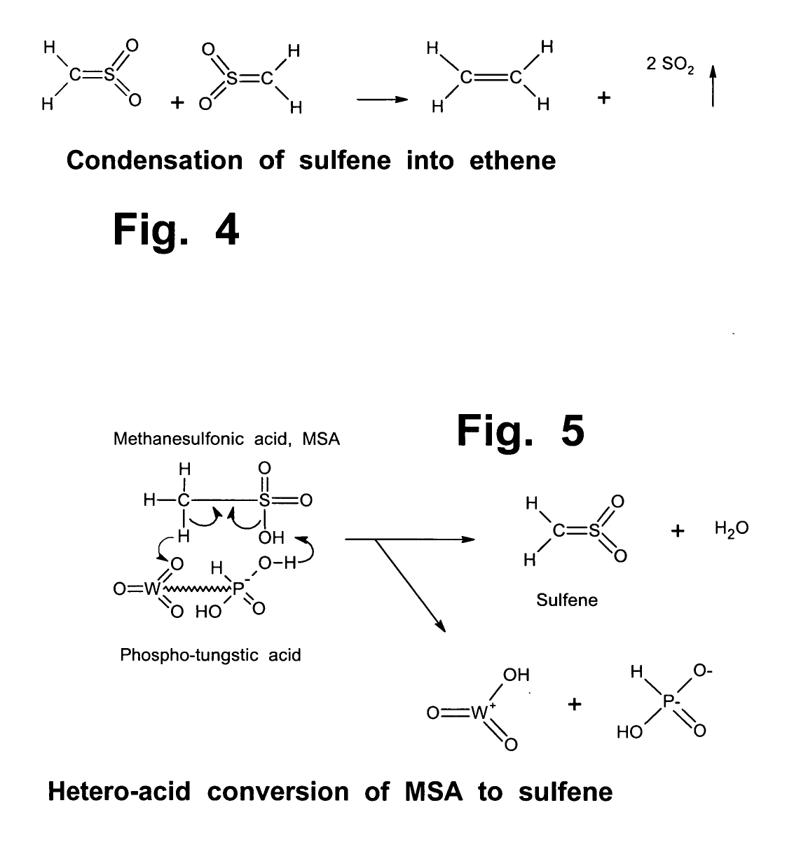

If the sulfane that is generated in this manner is converted into ethylene, as shown in FIG. 4, that reaction will be extremely rapid and exothermic, and it will release energy, calculated as a ΔH value −54.09 kcal/mol for each mole of ethene created. However, it requires two moles of sulfene to create a mole of ethene. Therefore, the endothermic (energy-consuming) ΔH value of 34.83, for creating a mole of sulfene from MSA, must be doubled, when overall energy requirements are considered. This leads to an endothermic value of 69.66 for creating two moles of sulfene, followed by a −54.09 “partial payback” of energy that is released when the two moles of sulfene are combined to form one mole of ethylene.

That leaves a net energy consumption requirement of 15.57 kcal/mole for producing ethylene from MSA; however, the calculations do not stop there. Two moles of SO₂ will be released by the sulfene-to-ethylene reaction, and the SO₂ must be oxidized back into SO₃, which will be recycled back into the reactor that is converting methane into MSA. The oxidation of SO₂ to SO₃ is highly exothermic, releasing −20.86 kcal/mole, and that value is doubled (to −41.72 kcal) when two moles of SO₂ are oxidized. Therefore, the entire system, which starts with MSA and ends with ethylene and SO₃, is exothermic, and yields a net energy release of −26.15 kcal/mole, when two moles of MSA are converted into one mole of ethylene and two moles of SO₃.

These computer-modeled numbers were calculated based on a simple and non-optimized solid support, involving nothing more than hydroxy groups on a silicate material. Now that a pathway has been disclosed for converting methane gas (which is wasted by flaring and reinjection, at rates of roughly $100 million worth of methane, every day) into ethylene (a highly valuable olefin), via MSA and sulfene intermediates, those who specialize in designing and testing solid catalytic materials (including silicates, cordierite, mullite, silicon carbide, etc.) can identify and optimize various combinations of supports and activating agents that are likely to create solid catalysts that can increase the yields, selectivity, processing rates, and other desired traits of these reactions.

### Candidate Pathway #2: Bifunctional (Friedel-Crafts) Catalysts

Anyone attempting to develop improved catalysts for use as disclosed herein should also consider “bi-functional” catalysts that use symphoric, anchimeric, and/or “neighboring group” effects to increase their ability to manipulate MSA. Such catalysts are also referred to as Friedel-Crafts catalysts (described in various patents and articles, such as U.S. Pat. No. 2,334,565), as acid-base (or acidic-alkaline) catalysts, or by similar terms. These catalysts presumably should be affixed to solid supports, to enable them to be retained inside a reactor vessel while large quantities of gas or liquid are being pumped through the vessel. Therefore, these types of catalysts can be regarded as a subset of Candidate Pathway #1, discussed in the preceding subsection, and they build upon and extend the teachings of that prior subsection.

In layman's terms, bifunctional catalysts can be regarded as “two-handed” catalysts. By way of analogy, most people cannot securely grip and hold a basketball with just one hand; however, nearly anyone can do it, using two hands. Similarly, it is easier (and faster) to cut a piece of steak, or an uncooked carrot or potato, if someone uses one hand (either with or without a fork) to hold the food stationary, while using the other hand to hold and use a knife.

In analogous ways, “two-handed” catalysts can attract, grasp, and manipulate some types of molecules more rapidly, efficiently, and securely than catalysts having only one type of active site or group. This is especially true with a molecule such as MSA, which has methyl and sulfonic domains that are very different from each other.

Therefore, rather than using a silicate support that only has hydroxy groups as the active sites (as described in the first candidate pathway, above), more efficient catalysts can be developed by providing a catalytic surface with two or more types of functional groups, allowing one type of group to attract and interact with the sulfonic domain of MSA, while a different group attracts and interacts with the methyl domain.

In general, most bi-functional catalysts use either or both of the following: (1) two distinct mechanisms that occur in sequence, usually quite rapidly (such as within nano-, micro-, or milliseconds); and/or, (2) anchimeric, symphoric, or neighboring group effects, in which a partial shift in one part of a molecule enables and promotes a secondary shift in another part of the molecule. In either case, an initial reaction or shift usually avoids or minimizes some type of transitional barrier that otherwise would hinder or block the second part of the desired reaction.

This approach to using bi-functional catalysts can be illustrated by a description of how a “hetero-polyacid” compound, such as tungsto-phosphoric acid (also called phospho-tungstic acid) can function in a desired manner, in a liquid solution. This teaching can then be adapted for use with immobilized catalysts (such as tungsten oxides) on solid supports.

Hetero-polyacid compounds usually are formed by combining two or more types of salts, then using a strong acid to acidify the salt mixture. For example, tungsto-phosphoric acid can be created by: (1) mixing a tungsten salt such as sodium tungstate dihydrate, Na₂WO₄.2H₂, with a sodium phosphate hydrate such as Na₂HPO₄.12H₂O, in distilled water; (2) adding concentrated hydrochloric acid, slowly and with vigorous stirring; (3) evaporating the water under a vacuum; (4) extracting tungsto-phosphoric acid from the residue, using a solvent such as ethyl alcohol; and, (5) removing the solvent under a vacuum, to provide a crystalline acid. This mixture will contains various species that can be represented as WOₓ (W is the symbol for tungsten) and H_{y}PO_{z}, where x, y, and z are variable numbers (usually but not always represented as integers). If desired, the acid mixture can be represented by one or more dominant species, such as WO₃.H₃PO₃. This compound is sold by Alfa-Aesar (www.alfa.com). Additional information is contained in sources such as U.S. Pat. No. 3,974,232 (Aizawa et al 1976, assigned to Toray Industries), which describes both (i) the dehydration of cyclohexanol into cyclohexene, and (ii) the use of other hetero-polyacid compounds, such as tungsto-silicic acid, molybdo-silicic acid, and molybdo-phosphoric acid.

If tungsto-phosphoric acid is mixed with MSA under suitable conditions and pressures, at least some of the MSA is likely to react with the acid in a manner shown in FIG. 5. In one part of the reaction, tungsten trioxide (WO₃) will extract a hydrogen atom from the methyl portion of the MSA. In a second part of the reaction, phosphoric acid will donate a hydrogen to the sulfate group on the MSA, causing it to become an ionic group with an extra hydrogen proton, —SO₂OH₂⁺. The —OH₂ group will then leave, as water (i.e., as steam, if the reaction is carried out at high temperature), leaving behind an ionic sulfate group, —SO₂⁺. This generates a transitional MSA ion that is likely to rearrange into sulfene, while releasing water, as shown in FIG. 5. These reactions also allow positively-charged tungsten ions (WO₃H⁺) and negatively-charged phosphate ions (H₂PO₃⁻) to reassociate, through ionic attraction, to reform a tungsto-phosphoric acid mixture. Accordingly, this compound functions as a catalyst, rather than as a consumed reagent.

To render it useful for continuous-flow processing of liquids or gases, bifunctional catalysts can be immobilized on solid supports, to prevent the catalyst from being washed out of a reactor by the gas or liquid flowing through the reactor. Immobilization of a bi-functional catalysts can be accomplished by using and/or adapting various synthesis routes that are already known to skilled experts who specialize in creating and testing new types of zeolites and other semi-permeable catalysts. As one example, the “SAPO” class of zeolite catalysts already contains phosphorus atoms, in an aluminosilicate matrix. Therefore, an activating agent can be used to donate tungsten atoms or groups in a manner that will coat the accessible surfaces of a SAPO material, in a manner that will create immobilized groups that are similar to tungsto-phosphoric acid mixtures as described above.

Alternately, if desired, a donor compound can be added to a reagent mixture that is being used to synthesize a porous catalyst. However, this approach tends to make less efficient use of an expensive “dopant” compound, since much of the dopant is likely to be inaccessible, inside the final material, rather than merely coated onto accessible surfaces.

Examples of hydrocarbon processing using bi-functional catalysts are provided in numerous published articles. As one example, scheme III shown on page 427 of Olah 1987 represents two sequential reactions that are triggered, first, by an acidic domain of a catalyst, and second, by an alkaline domain of the same catalyst material. Olah et al 1984 (entitled, “Onium Ylide Chemistry: 1. Bifunctional Acid-Base-Catalyzed Conversion of . . . ”) contains more information on this subject.

It should also be noted that many metal oxide catalysts (such as vanadium oxide catalysts, platinum oxide catalysts, etc.) act as bi-functional catalysts, in which a vanadium, platinum, or other metal atom interacts with one domain of a compound being treated, while one or more oxygen atoms bonded to the metal atom interact with a different domain of the compound being treated.

Accordingly, since MSA has two different domains that will respond differently to different catalytic sites, bi-functional catalysts hold exceptionally good promise for converting MSA into sulfene, in ways that adapt and extend the teachings set forth above, concerning solid-supported catalysts in general.

### Candidate Pathway #3: MSA Methyl-Ester Pathway

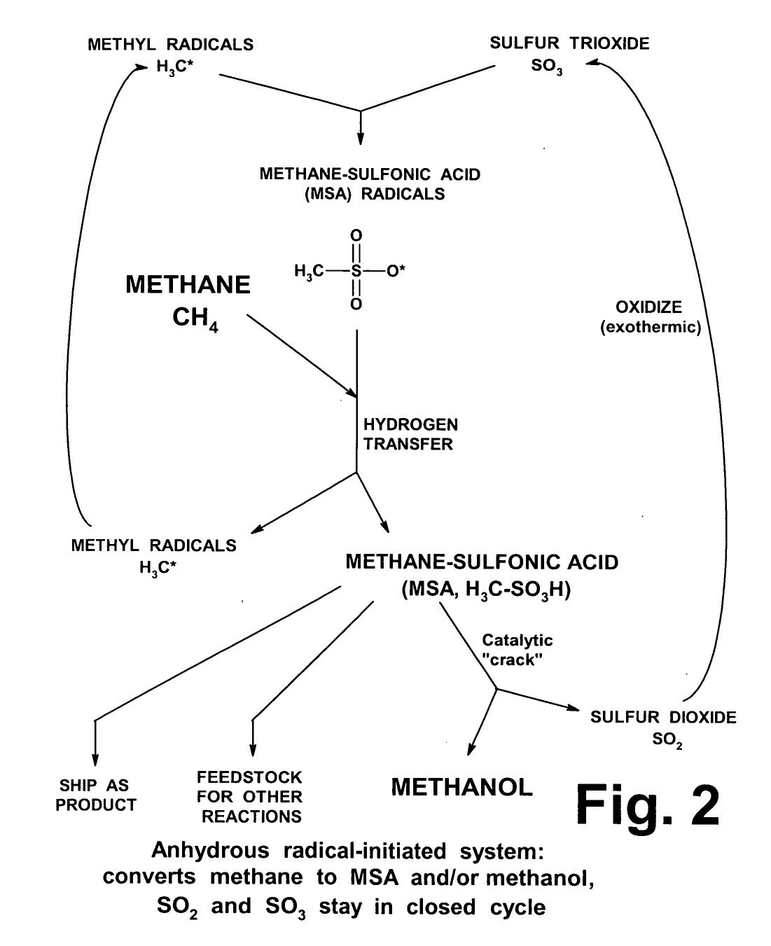

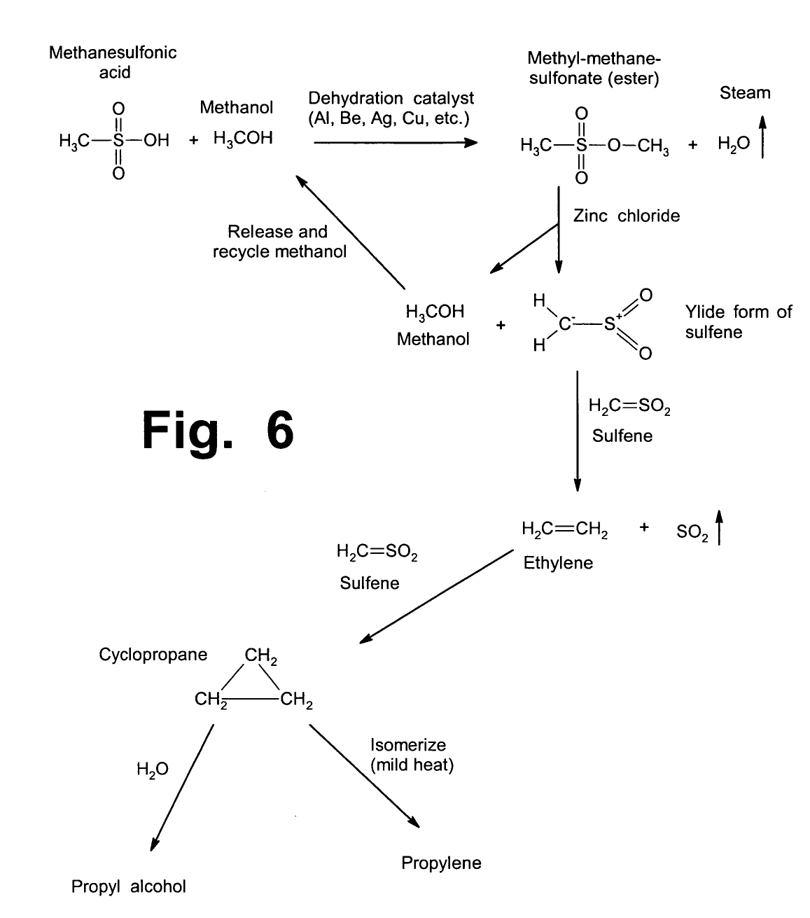

During the course of the laboratory research that confirmed the computer-modeled MSA-forming pathway shown in FIG. 2, a question arose as to whether the MSA was (or might be, under certain conditions) contaminated by a methyl-methanesulfonate ester, with a structure shown at the top of FIG. 6. That question triggered some additional analysis and computer modeling, as well as a careful rereading of every patent issued to Snyder and Grosse in the early 1950's.

That work eventually resulted in a postulated pathway that (according to the computer modeling results) appears to offer an improved pathway to sulfene, with lower thermodynamic hurdles than candidate pathway 1, above.

This candidate pathway is illustrated in FIG. 6. It is commenced by reacting MSA with methanol, in the presence of a dehydration catalyst (various metals, such as aluminum, beryllium, silver, and copper offer candidates for evaluation for such use), in order to deliberately convert the MSA into the methyl-methanesulfonate (MMS) ester. This dehydration reaction will be carried out at an elevated temperature, and the water that is released will be removed from the reactor, as steam. Additional information that can shed additional light on this dehydration reaction, and on catalysts that can promote it, can be gleaned from U.S. Pat. No. 2,553,576 (Grosse & Snyder, 1951).

The MMS ester compound is then treated with a highly polarized metallic salt, such as a zinc halide, such as zinc chloride, ZnCl₂, which can act as a “Friedel-Crafts” catalyst. As suggested by various comments in U.S. Pat. No. 2,492,984 (Grosse & Snyder, 1950), when those comments are combined with and interpreted in the light of a modern understanding of “ylide” compounds and structures (which are briefly summarized below), the metal halide will effectively pry off the —OCH₃ (methoxy) group from the sulfate group. This methoxy group will then react with a hydrogen proton, which will be available from one or more other sources in the reactor vessel (such as from a methyl group on MSA or MMS, which will be releasing protons as they convert from an H₃C— methyl group into an H₂C═ sulfene group). The reaction of the methoxy group with a hydrogen proton completes the re-formation and release of methanol, which is recycled back into the dehydration reactor.

The release of methanol, from the MMS ester, leaves behind an “ylide” form of sulfene, as the residue. Various candidate solvents can be tested, to determine which solvent(s) can maximize the yields of this reaction. Tetrahydrofuran, dimethylsulfoxide, and other candidate solvents can provide a range of polarity levels, which will merit evaluation for such use. In general, the preferred solvent selected for a particular manufacturing facility is likely to depend on the operating temperature that is used, at that site. Solvents with lower polarity levels can be used to slow down SO₂ removal, in ways that can be used to control reaction kinetics, to maximize desired yields.

The sulfene ylide compound will react with sulfene to form ethylene, a valuable olefin used to make plastics and polymers. These reactions are complex, involving various types of pi and sigma bonds. They potentially involve four-member “dimer” intermediates that will decompose at suitable temperatures, enabling the SO₂ groups to act as leaving groups while ethylene is formed (for more information on such dimers, chapter 17 by King and Rathore, from Patai and Rappaport 1991, and Arnaud et al 1999, should be consulted. However, computer modeling indicates that sulfene is sufficiently electronegative and reactive that it is likely to avoid any thermodynamic barriers, and may bypass any such dimer formation. This modeling further supports the conclusion that sulfene may be one of the most active and effective methylene transfer agents ever identified, and may open up exceptionally efficient pathways to various types of hydrocarbon chemistry that have not previously been available. In addition, various options can be evaluated for controlling the levels of sulfene reactivity, including, for example: (i) carrying out such reactions at low temperatures, under reduced pressures, and/or in the presence of various solvents that will help sustain reactions at lower rates; and, (ii) modifying MSA, prior to dewatering it, in ways that may, for example, modify one or more of the oxygen atoms on the sulfonic group.

If the ethylene remains in solution while additional sulfene is being formed, at least some of the ethylene is likely to react with sulfene, to form cyclopropane. This is a relatively unstable molecule, due to the fact that its bond angles are stressed, at 60 degrees, compared to the normal 109.5 degrees for unstressed bonds in an aliphatic chain. Accordingly, cyclopropane can be isomerized by steps such as mild heating, to form propylene (a valuable olefin that is easier to handle and transport than ethylene), or it can be reacted with water to convert it into propyl alcohol, which makes a very good gasoline additive or substitute, with an energy density higher than methanol or ethanol.

While the modeling data generated to date rely on various assumptions, and are not regarded or represented as final and authoritative, a comparison of certain numbers for different pathways that were generated using fairly consistent assumptions can provide some indication of the relative merits of the MMS-plus-methanol pathway, compared to the first pathway described above, using a silicate support with exposed hydroxy groups. Under standard conditions, at 300 degrees Kelvin, the dehydration reaction (to reach the MMS compound and release steam) showed an endothermic ΔH value of +11.7 kcal/mol, and a Gibbs free energy value of ΔG 12.3 kcal/mol, and the reaction that released methanol and formed sulfene ylide showed an endothermic value of ΔH=+23.1 kcal/mol, and a Gibbs free energy value of ΔG=11 kcal/mol. For comparison, MSA conversion on silica with hydroxy groups showed an endothermic value of ΔH=+34.8 kcal/mol, and a Gibbs free energy value of ΔG=23.3 kcal/mol.

The fact that all three of the candidate pathways disclosed above, for making sulfene, regenerate their starting materials, and treat those materials as catalysts rather than consumed reagents, deserves attention, especially when compared to pathways reported in the prior art, which could generate sulfene only by consuming reagents and generated unwanted byproducts (as one example, Prajapati et al 1993 describes a method of generating sulfene which consumed SOCl₂, and generated hydrochloric acid). Since catalytic processes are generally superior to processes that generate unwanted wastes (especially acidic and/or salt wastes), it is believed that this invention discloses a new and useful improvement in synthesizing sulfene, regardless of what is subsequently done with the sulfene.

With regard to all of the candidate pathways listed above, the enthalpy calculations in Table 1 merit consideration, and certain factors that dwell within those numbers should be noted in specific. The conversion of MSA into sulfene is endothermic, and requires energy to drive it, at a ▴H value calculated as 34.83 kcal/mole. However, the ▴H value for subsequently converting sulfene into ethene is exothermic, and releases 54.09 kcal/mol. The net release of energy is about 20 kcal/mol, to move from MSA to ethene. This is a crucial factor that sits at the heart of this aspect of the invention, and all of the candidate pathways disclosed herein should be regarded as ways to drive the MSA dewatering reaction over an initial “hump”, so that the reaction can reach the downhill slope of the energy curve, where it will continue on its own, releasing substantial net energy.

Accordingly, a proper understanding of this invention herein should not focus solely on converting methane or MSA into sulfene, since sulfene is an unstable high-energy intermediate. Instead, this invention discloses a useful pathway for converting methane or MSA into olefins or other valuable and useful products, by passing through sulfene, an intermediate that effectively provides a pathway for making olefins and other materials, with lower thermodynamic barriers than other alternative pathways.

It also should be emphasized that the candidate pathways described above are not regarded as exhaustive or exclusive. Instead, other candidate pathways for reaching sulfene, or for reaching other useful intermediates that can be converted into olefins or other valuable products, are likely to be recognized by those skilled in the art, after the chemical pathways and commercial prospects for converting stranded and wasted methane into MSA, then sulfene, and then ethylene, have been disclosed.

### Use of Sulfene and Other Ylides; Methylene Transfer Agents

In addition to being useful for the manufacture of ethylene, sulfene may be potentially useful as a “methylene transfer agent” (MTA) in various other situations. This potential utility will be limited by the tendency of sulfene to react with itself, rapidly and exothermically, to form ethylene; nevertheless, by controlling reaction conditions such as temperatures, pressures, and ratios of reagents, it may be possible and practical to induce sulfene to react with various other compounds, in various ways and at commercial levels.

Cyclopropane offers a good example, since it can be formed by reacting ethylene (formed by condensing sulfene, or by any other known method) with additional sulfene. If a fixed quantity of sulfene is placed or created in a closed reactor, much of the sulfene is likely to form ethylene, fairly rapidly; then, as the ratio of ethylene to sulfene in the reactor rises, the remaining sulfene will become more likely to react with the growing quantity of ethylene, than with the dwindling quantity of remaining sulfene. In a similar manner, if a gaseous ethylene feedstock is continuously fed into a reactor along with a limited quantity of sulfene, at least some of the sulfene will react with the ethylene.

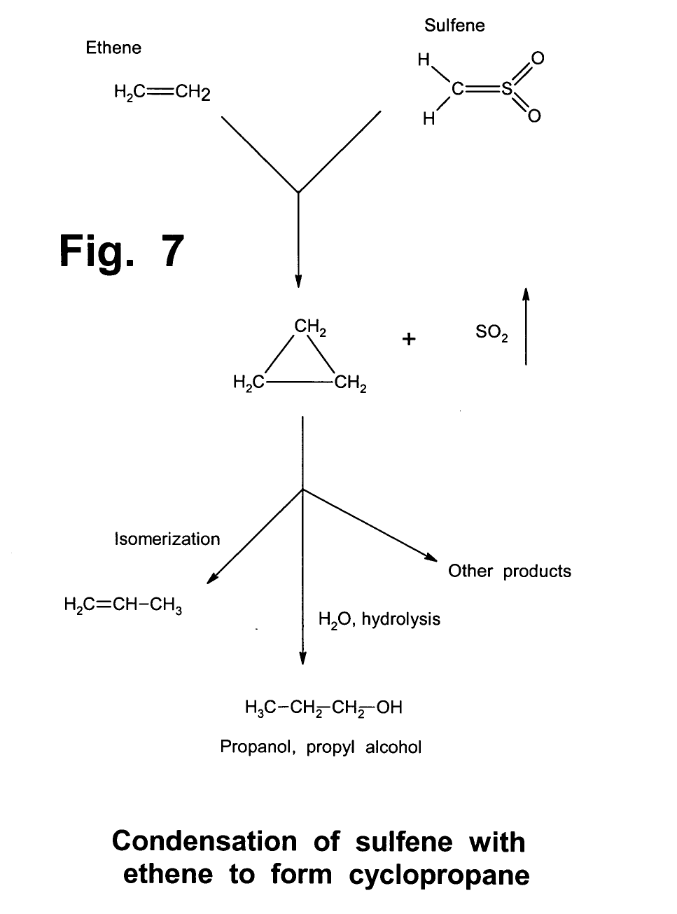

Accordingly, FIG. 7 depicts a reaction pathway for using sulfene to convert ethylene into cyclopropane. Briefly, when a molecule of sulfene contacts a compound having a double bond, the sulfur dioxide group from the sulfene will leave, and the methylene group (which has two unshared electrons, and which can be represented as either H₂C: or —CH₂—) will react with the double bond, in a way that generates a triangular structure, as shown in FIG. 7. If the reagent with the double bond comprises ethylene, the triangular product will be cyclopropane, which is useful and valuable because it is highly reactive, due to the fact that its bonds are stressed at 60 degree angles in a planar structure (by contrast, the conventional bond angle in alkane molecules is 109.5 degrees).

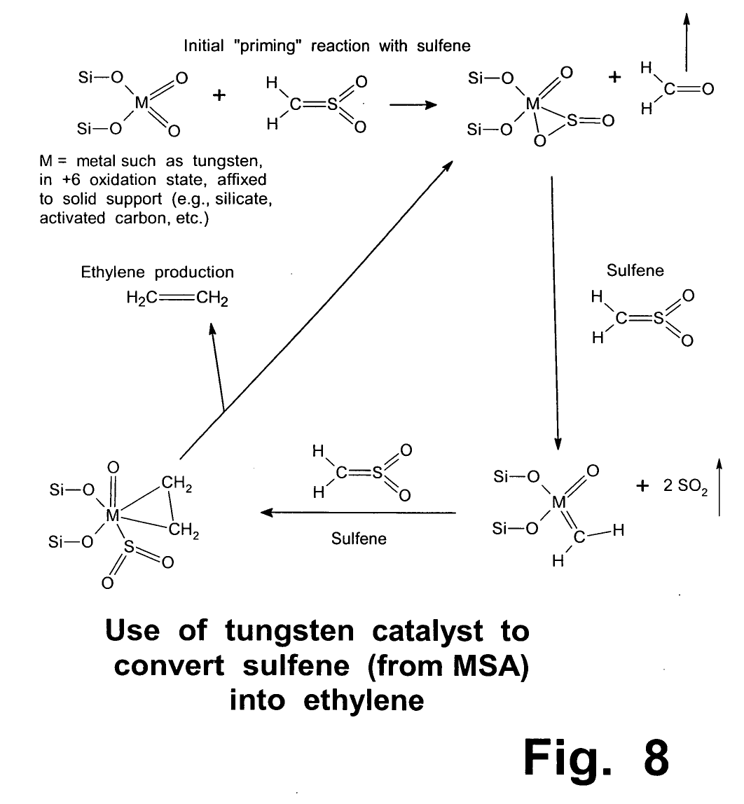

If a limited and appropriate amount of energy is put into cyclopropane, the cyclopropane can overcome a transitional energy barrier and undergo an isomerization reaction, as shown in the lower portion of FIG. 8, to form propylene (also called propene), H₂C═CH—CH₃. This is a valuable olefin, useful for making polypropylene and other plastics and polymers. Since propylene is larger and heavier than ethylene, it is less volatile, and more inclined to behave as a liquid rather than a gas, at temperatures and pressures that can be achieved more easily and at less expense than required for ethylene. Propylene can be stored and transported as a liquid, using tanks that can operate at lower operating pressures and/or warmer temperatures than required to store or transport ethylene as a liquid. Therefore, ethylene-to-propylene conversion, using sulfene as shown in FIG. 7, may have important commercial implications.

Alternately, cyclopropane can be reacted with water, in a manner that breaks one of the stressed triangular bonds, in a way that creates propyl alcohol (also called propanol). This reaction can be referred to either as hydrolysis (since one of the carbon-carbon bonds is broken), or as hydration (since the components of a water molecule are being added to the cyclopropane). Propyl alcohol is a clean-burning fuel, which can be used as a gasoline additive or substitute with higher energy content than methanol or ethanol, and it has other valuable uses as a chemical feedstock, skin disinfectant, etc.

Prior to these discoveries and disclosures, sulfene has not received any close or careful attention by chemists, for three main reasons: (i) it is highly reactive, unstable, and short-lived; (ii) it is very difficult to store or transport, and, (iii) it was difficult to synthesize, and the known methods created serious problems of toxic and hazardous wastes.

However, if efficient and economic methods for manufacturing sulfene from stranded methane (which currently is being wasted and destroyed in huge volumes, every day) are made available by the discoveries and disclosures of the Applicant, sulfene will deserve and receive much more attention and analysis.

In particular, sulfene can be regarded and used as a dipolar compound that has both “super-nucleophile” and “super-electrophile” traits. This gives it an exceptionally potent ability to react with double bonds, in ways that can avoid the destruction and elimination of the double bonds.

Indeed, in addition to regarding sulfene as a dipolar compound (due to the differences between the CH₂ component and the SO₂ component), a methylene radical, by itself, can be regarded as a dipolar and bi-functional agent that is both a super-nucleophile, and a super-electrophile. On one level, a methylene radical has two extra and unpaired electrons exposed on its surface, and those electrons will aggressively seek out and bind to the positively-charged nucleus of another carbon atom. That makes methylene radicals highly potent nucleophiles. However, at the same time, a methylene radical is missing two electrons from its valence shell, and it will aggressively seek out and bind to an electron-rich structure which can help it fill those gaps (such as a double bond, in an olefin molecule). That makes methylene radicals highly potent electrophiles.

These combined traits are believed to make sulfene a highly potent “methylene transfer agent”, which can insert —CH₂— groups into various types of compounds. In one particular type of reaction that is likely to become of scientific and commercial interest, it is believed that sulfene will be able to insert methylene groups into olefins, without destroying the double-bonded constituents of the targeted olefins. As such, it is believed to be able to convert propene into butene, butene into pentene, pentene into hexene, etc., by chain-lengthening reactions in which the sulfene is most likely to react with the electron-rich double bond, in each step of the reaction. The initial step in a “methylene insertion” reaction will create a three-membered ring, comparable to a cyclopropane molecule that has a “tail” attached to one of the three carbons. The ring having three carbon atoms (with stressed bonds, having bond angles of 60 degrees) can then be induced (such as by moderate heating) to cross a relatively low transitional energy barrier, in a way that isomerizes the three-member ring to form an “alpha” olefin, with the double bond positioned between the first and second carbon atoms in the chain. This isomerization form is believed to be preferred over a 2,3-olefin formation, because the #3 carbon atom in a three-membered ring (i.e., the carbon atom that has a hydrocarbon “tail” attached to it) will be less electron-rich, and less likely to participate in the formation of a double-bond.

By screening and optimizing different zeolite or other porous catalyst formulations, and by manipulating the use of “seeding” compounds that can serve in a manner comparable to “condensation nuclei”, this approach can be used to manufacture liquid mixtures that will be comparable to “fractions” that can be obtained by distillation or other conventional hydrocarbon processing, with sufficient quality and consistency to enable their use as gasoline or other fuels, or as fuel additives, blending agents, etc., without requiring distillation or other purification (although distillation or other purification can be provided, if desired, to increase the purity and value of any resulting product(s)). In some cases, it likely will also be possible to generate liquids that are sufficiently enriched in one or more dominant compounds that they can be used as chemical feedstocks, either without additional purification, or after purification by means such as by distillation, molecular sieves, etc.

It also is likely and anticipated that methods will become known and available (such as by manipulating temperature, pressure, and time conditions, catalyst formulations, and/or condensation nuclei) for manufacturing various different categories of liquid hydrocarbons, including straight-chain alkanes, branched alkanes, alkenes (also called olefins), cycloalkanes and cycloalkenes, aromatics, and possibly even substituted hydrocarbons (such as halogenated or oxygenated derivatives, etc.).

As a demonstration of that potential, some of the initial tests carried out to date indicate that under some conditions, methane reagents that have passed through MSA and then sulfene intermediates have generated liquid hydrocarbon mixtures that qualify as naphtha-type mixtures (generally defined as a crude oil fraction that can be obtained by distillation, containing molecules within the C4 through C12 range). This result indicates that this approach may offer the most efficient and economical method ever discovered to date, for converting methane gas into liquid hydrocarbons that can be used for high-quality gasoline or other fuels, or for chemical feedstocks (which can be especially valuable if a double bond remains present in the hydrocarbon molecules).

In addition, early tests involving different conditions have indicated that under some conditions, sulfene-containing preparations can generate solid polymeric materials. Such plastic and/or polymeric materials, when manufactured in this manner at methane-producing sites, have a wide range of uses; for example, they can be stored and transported in particulate form, in ways that allow them to be melted and molded into desired shapes, at a factory.

Because of the reactivity of sulfene, it is likely that most commercial-scale reactions involving sulfene will generate a mixture of products, rather than a single relatively pure product. However, various types of separation processes (such as distillation, centrifugation, molecular sieves, etc.) can be used to separate mixed product streams into relatively pure product fractions, if desired. Accordingly, preferred product mixtures or purified product streams will depend more heavily on economic factors and preferences than on technical constraints. It should also be noted that the presence of a substantial quantity of cyclopropane and/or propene, in a liquid or gaseous mixture that also contains ethene, is likely to lower the vapor pressure of the ethene, in ways that will make it more efficient and economic to transport a mixture in liquid form. Similar effects occur with other hydrocarbons, including “liquified natural gas” (LNG) mixtures, in which butane and/or pentane effectively help to “solubilize” propane in a liquid mixture. This allows large quantities of propane to be stored and transported, in LNG mixtures, at pressures substantially lower than would be required for propane alone.

Therefore, the ability to use sulfene in various types of chemical synthesis and manufacturing operations, and the economic, technical, and commercial possibilities that will become available if reactions that pass through sulfene as a reactive and unstable intermediate can be efficiently and economically carried out in large quantities by the methods disclosed herein, appear to have the potential to open up a number of new pathways and fields, in organic chemistry. These options and opportunities will merit careful evaluation, after the disclosures herein have been revealed to chemists who are skilled in these branches of organic chemistry.

### Ylides and Carbanions

Chemists interested in sulfene chemistry should understand (or at least study) compounds called ylides and ylids, and so-called “Wittig reactions”, named after Georg Wittig, a German chemist who won the Nobel Prize in 1979. A complete analysis of ylid and ylide chemistry is beyond the scope of this application; however, they are discussed in detail in various review articles (e.g., Li et al 1997 and Lakeev 2001), and full-length books (e.g., Trost 1975, Clark 2002, and Bertrand 2002).

Very briefly, ylides and ylids that are of interest herein will have a “carbanion”, a term that combines “carbon” with “anion”. This refers to what is, in effect, a carbon atom with an unshared electron pair. This unshared electron pair is created by positioning the carbon atom next to a positively-charged “hetero-atom”, which will donate one of its electrons to the carbon atom (a more complete description of this electron shift requires an analysis of electron valence shells, “p” and “d” orbitals, pi bonding, etc.). In most cases of commercial interest, the heteroatom will be sulfur, nitrogen, or phosphorus, although some chemists regard oxygen as also having sufficient strength to form compounds that can behave as ylides under at least some conditions).

Ylides and other compounds that contain negatively-charged, electron-rich “carbanions” are relevant to the manufacture of olefins, for the following reason: if two molecules that contain electron-rich “carbanions” react with each other, the electron-rich “carbanions” in the reagent molecules are likely to form an electron-rich double bond, between the two carbon atoms, in a new molecule created by the reaction, while the positively-charged heteroatoms act as leaving groups.

Examples of ylide chemistry are discussed in Corey et al 1965, which addresses two particular ylides: dimethylsulfonium methylide, (CH₃)₂S═CH₂, and dimethyloxosulfonium methylide, (CH₃)₂S(O)═CH₂ (the parentheses around the oxygen atom, in the oxosulfonium ylide, indicate that the oxygen is double-bonded to the sulfur atom, rather than being positioned between the sulfur and carbon atoms). In both of those ylide compounds, the bond between the sulfur atom and the carbon atom can be written in any of three ways (often called “canonical” forms, comparable to a musical piece such as “Pachelbel's Canon”, in which the same melody is played repeatedly, but in slightly different ways). One written version depicts a standard double bond, such as R₁R₂S═CH₂. A second written version depicts a single bond with charge indicators, R₁R₂S⁺—C⁻H₂. A third written version combines those two formats, and depicts a double bond with charge indicators, R₁R₂S⁺═C⁻H₂.

The term “resonating structure” is often used to describe electron configurations that cannot be cleanly represented as one particular form. Most commonly, resonating (or resonant) electron structures can have either or both of: (i) two distinctly different forms which will shift back and forth, to coexist with each other, in equilibrium; or, (ii) a quasi-stable intermediate form, located somewhere between the two ends of the continuum, and having some combination of mid-point properties. Resonating electron structures are fairly common in chemistry, and are used to explain a wide variety of semi-stable molecules, including carbon monoxide, sulfur dioxide, and molecules that shift back and forth between “tautomeric” forms (such as sugar molecules, which shift back and forth between rings, and straight chains).

One set of teachings in Corey et al 1965 is worth noting. When Corey et al used the reagents described in the lower right column of page 1363 to synthesize dimethylsulfonium methylide (which Corey et al described as CH₃)₂S⁺═C⁻H₂, shown as compound XIII in the left column of page 1356), the temperature of the reaction mixture rose slightly, and released a gas, most of which evolved within 5 minutes. That gas was passed through a bromine solution, and the resulting gas was analyzed and found to be ethylene dibromide. This indicated that the gas, released by spontaneous exothermic decomposition of Corey's methylide compound (which contained the S⁺═C⁻ ylide structure) was ethylene, and the dimethylsulfide group of the ylide compound acted as a leaving group. That report provides additional support for the assertion that sulfene will spontaneously react with itself, in a way that releases ethylene.

The disclosures herein also suggest that dewatering of other alkane-sulfonic acids (such as ethanesulfonic acid, propanesulfonic acid, etc.), using agents and methods such as described above to create sulfene analogs or other ylide compounds, can provide useful approaches to manufacturing other types of longer and heavier olefins.

Accordingly, the disclosures herein can be combined with additional disclosures (already published in the art) involving ylides, ylids, and Wittig reactions, in ways that will enable commercial and industrial adaptation of sulfene and sulfene-analog chemistry for use with additional types of ylids and ylides, in ways that will become apparent to those skilled in that particular field of chemistry, after they have analyzed and evaluated the disclosures herein.

### Use of Metal Catalysts to Increase Ethylene Yield and Purity

As mentioned above, ethylene (a feedstock for manufacturing plastics and polymers) can be formed when sulfene reacts with itself. However, because of the aggressive reactivity and instability of sulfene, other byproducts also can be formed. Accordingly, this section discloses the use of certain types of catalysts for improving the selectivity and yield of ethylene forming reactions.

One advantage of this approach is that suitable metal catalysts appear to be capable of catalyzing two different reactions, occurring in very rapid succession at essentially the same site. The first reaction converts MSA, a relatively stable compound, into sulfene, an unstable and short-lived intermediate. Without delay, and without requiring any diffusion or other molecular transport of the sulfene molecules to a different site, the sulfene can then react with the same or nearby catalytic atoms on the surface of the same catalyst (which can be affixed to a solid material, such as packed or stirred beads, a porous monolithic material, etc.) in a manner that will rapidly create and release ethylene, the desired product, as a gas. This type of processing, which will create and then consume sulfene in a highly efficient “straight-through” pathway, is often called a “single pot” reaction by chemists, and it can be highly useful and efficient. Accordingly, the preparation of a tungsten oxide catalyst, and the use of that catalyst in a highly efficient reaction that converted MSA into ethylene, are described in the examples below, and those tests confirmed that ethylene production from MSA had an apparent selectivity of about 95%.

This catalytic pathway uses metal atoms that can be driven to a +6 oxidation state without using extreme conditions. The exemplary candidate metal catalyst that has been computer-modeled to obtain the favorable results described below, and that also has been experimentally tested with very good results, is tungsten. Other candidate metals that can be driven to a +6 oxidation state, and that can be evaluated for use as disclosed herein, if desired, include vanadium, ruthium, etc.

This invention does not depend on any particular reaction pathway; instead, it depends on the disclosure of a practical means for selecting and making catalytic materials that can help efficiently manufacture desired products. Nevertheless, a potential reaction pathway with apparently favorable thermodynamics has been determined, by computer modeling, using the Amsterdam Density Functional program (release 2.3.3, by Scientific Computation and Modelling (www.scm.com), described in detail in te Velde et al 2001). That candidate reaction pathway is illustrated in FIG. 8 herein.

As shown in FIG. 8, the catalytic material uses a metal atom (represented by M in drawing, and exemplified by tungsten) that is driven to a +6 oxidation state (which can be done by oxidation treatment of a preexisting surface, by selection of suitable tungsten oxide reagents for making the catalyst, etc.). To render the process practical and economic for large-scale industrial operations, the catalytic metal should be affixed to a solid support that can be trapped and retained within a reactor vessel. A silicate support material is shown in FIG. 8; however, since the support is essentially inert, other types of solid support (such as activated carbon, or various mineral or ceramic materials used to make porous monolith materials) can be evaluated for such use if desired, and any physical configuration of interest (such as porous monoliths, packed or stirred beads or other particulates, etc.) also can be evaluated.

When the tungstate catalytic surface (shown in the upper left corner of FIG. 8) is first contacted by sulfene, it will lead to the formation of a first intermediate with a stressed three-membered “tungstate-sulfoxy” ring that contains tungsten, sulfur, and oxygen, shown near the upper right corner of FIG. 8. This can be regarded as a “priming” operation. It will release some quantity of formaldehyde, but only during the “priming” step; formaldehyde production will not continue, when the main cycle of the reaction proceeds. Formaldehyde is a valuable chemical reagent, and it will condense into a liquid at a relatively low temperature, thereby allowing simple removal (using a device such as a liquid trap) without requiring distillation or other complex processing or additional energy input, while the ethylene (or possibly other products) remains gaseous and can be removed in relatively pure form from a different reactor outlet. Alternately, since formaldehyde is a valuable byproduct, the reaction disclosed in FIG. 8 (or analogous reaction pathways) can be adjusted and adapted in ways that will generate larger and continuous quantities of formaldehyde. For example, computer modeling indicates that if oxygen is added to the catalytic material while it has a CH₂ group bonded to the tungsten molecule, as shown in the structure in the lower right corner of FIG. 8, the formation and release of formaldehyde is likely to occur, in an exothermic reaction.

When a second sulfene molecule contacts the tungstate-sulfoxy intermediate (shown in the upper right corner of FIG. 8), the sulfene will effectively “knock off” the SO₂ group from the surface-attached catalytic intermediate, and in the same reaction, the sulfene will also release its SO₂ group. This causes the release of two molecules of SO₂. These will form gas bubbles, which will be released from the catalytic surface. The SO₂ gas can be collected and oxidized back into SO₃, which will be recycled back into the reactor that creates the MSA.

That sulfene reaction also causes a CH₂ group to become double-bonded to the tungsten atom on the catalytic surface, as shown in the lower right corner of FIG. 4.

This intermediate is contacted by yet another sulfene molecule, forming another unstable intermediate, as shown in the lower left corner of FIG. 8. This intermediate has a sulfoxide group, and two CH₂ groups (in a stressed ring structure), bonded to the tungsten atom.

The two CH₂ groups in the stressed ring structure will break away from the tungsten atom, in a way that forms a double bond between the two carbon atoms. This releases ethylene, in gaseous form, from the catalytic surface. When this occurs, the sulfoxide group attached to the tungsten atom also rearranges, in a way that reforms the sulfoxide ring structure shown in the upper right corner of FIG. 8.

As long as sulfene continues to be formed on or near that catalytic surface (due to an MSA dewatering reaction that is occurring on or near the same surface), the three-part cycle shown in FIG. 8 will continue to occur. In each cycle, two molecules of sulfene are consumed. The two SO₂ groups from the two sulfene molecules will act as “leaving groups”, and when released as SO₂ gas, they will be recycled back into the system, to make SO₃ for MSA formation. The two CH₂ groups from the two sulfene molecules will be bonded to each other, to form ethylene, which will emerge from the reactor in gaseous form.

As mentioned above and as described in Examples 4 and 5, this type of tungstate catalytic surface, on a conventional silicate support (in a porous monolith disc), was created and tested. It was shown to be highly efficient in converting MSA into ethylene, presumably via the sulfene intermediate as described above, using one or more pathways such as (or possibly similar to) the route shown in FIG. 8.

Briefly, the tungsten catalyst was created by immersing a conventional silica monolith into a solution of ammonium tungstate ((NH₄)₂WO₄) in water, then removing the disc and drying it, to remove all or most of the ammonium ions, leaving behind tungsten and presumably oxygen atoms. The immersion and drying process was repeated until the disc appeared to be saturated with tungsten, as evidence by a powdery residue in the bottom of the drying dish after the third cycle was completed.

Alternate methods are known or can be developed for coating tungsten (or other similar metals or metal oxides) onto surfaces of a solid support material. For example, other tungsten-containing compounds (such as sodium or potassium tungstate, as examples) can be evaluated for such use, and methods can be developed for rinsing and washing nonadsorbed sodium, potassium, or other ions out of or off of a solid support. Alternately or additionally, any other known or hereafter discovered coating method (such as “sputter coating” or other vapor-deposition methods, which can be promoted by inert gas flow through a porous material) can be used.

Similarly, an expensive catalytic metal or metal oxide can be incorporated into a solid catalytic material that is being manufactured. However, that approach, which distributes an expensive metal throughout the entire bulk of the catalyst, usually is more expensive than merely coating a very thin layer of an expensive metal onto the surface of a low-cost and relatively inert support material, such as silica or activated carbon.

It should also be noted that other transition metals that have various similarities to tungsten merit early evaluation for such use. Such metals include metals that are in certain “columns” of the periodic table, including:

(1) the 5b column, which includes vanadium (atomic symbol V). This column also includes niobium (Nb) and tantalum (Ta), but those two metals are rarer and more expensive than vanadium.

(2) the 6b column, which includes chromium (Cr), molybdenum (Mo), and tungsten (W);

(3) the 7b column, which includes manganese (Mn); it also includes technicium (Tc) and rhenium (Re), but those are relatively rare and expensive;

(4) the 8 column, which includes iron (Fe); it also includes ruthenium (Ru) and osmium (Os), but those are relatively rare and expensive.

In addition, the other “transition metal” columns in the period table (including the 4b column, which includes titanium, and the 9 through 12 columns, which are headed by cobalt, nickel, copper, and zinc and which include various soft and/or “noble” metals such as palladium, silver, platinum, and gold) also merit testing and evaluation for use as described herein, either for making sulfene, ethylene, or formaldehyde, or for carrying out the catalytic oxidation of SO₂ to SO₃.

Based on computer modeling to date, it is believed for now that the most promising candidate catalytic materials that deserve evaluation include metals that can assume a +6 oxidation state. This includes metals (such as iron, which normally will remain in a +2 or +3 oxidation state under most conditions) that can be forced or “driven” to a +6 oxidation state under the types of pressures and temperatures that are commonly used in oil and gas processing.

It also should also be noted that approaches using two or more different types of catalysts merit evaluation. For example, iron catalysts tend to be less efficient than other catalysts that contain more expensive metals. However, iron-containing catalysts also are relatively inexpensive, and they often can operate at high temperatures that will damage or destroy catalysts that use other, more expensive metals. Therefore, an economically preferred and useful processing system might use a first-stage reactor with an iron or other low-cost catalyst to achieve a “rough” or “first-pass” level of MSA-to-ethylene conversion (such as, for example, with yields in the range of about 40 to 80 percent), followed by a second-stage reactor containing a more expensive catalyst that can provide higher yields.

Various combinations of catalytic materials also can be mixed and included in a single reactor vessel. As examples of this approach, U.S. Pat. No. 6,596,912 (Lunsford et al 2003) and Makri et al 2003 describe the use of catalysts containing manganese and sodium tungstate, on a silica support, in a different types of processing (“direct” processing of methane, in which methane gas is directly contacted with a catalyst, at high temperatures, to form higher hydrocarbons).

Accordingly, this invention discloses an efficient catalytic method and material for converting MSA into an olefin. Those skilled in the art will recognize various ways to screen and optimize various alternate formulations of such metal catalytic surfaces, using (for example) the types of automated machines and methods that can be used to screen and evaluate dozens of candidate catalyst formulations in a single screening cycle, as described in articles such as Muller et al 2003, and other articles cited therein. Such devices use, for example: (i) reactors with multiple parallel tubes, each tube containing a different candidate catalyst, or (ii) titer plates with multiple wells, each well containing a candidate catalyst. When a reagent (such as MSA) is passed through or loaded into all of the tubes or wells, the product generated by each individual tube or well (and therefore by each candidate catalyst) is collected separately, and delivered to an automated analytical device, such as a mass spectrometer or chromatograph. The tubes or wells that created the highest yields of the desired compound can be identified, and the exact content of the catalysts in any tubes or wells that resulted in good and desirable yields can be identified and studied more closely. For example, the best-performing candidate catalyst from one round of tests can be used as a “baseline” or “centerpoint” material, in a subsequent round of tests that will use variants that resemble the best-performing catalyst from the previous round of screening. Those variants can include known and controlled compounds, having exact compositions; alternately or additionally, “combinatorial chemistry” methods and reagents can be used, to generate random or semi-random variants of a material that provided good results in an earlier screening test. Accordingly, these types of automated screening systems offer powerful and useful tools for rapidly identifying and/or improving porous catalyst formulations that can efficiently promote reactions that will convert MSA into ethylene (and possibly into propylene or other olefins).

Another factor worth noting involves catalytic agents that use symphoric, anchimeric, or “neighboring group” effects to enable “two-handed” manipulation MSA or other compounds. MSA has two very different domains, methyl and sulfonic. More potent and efficient catalysts might be developed, by providing a catalytic surface with two different types of functional agents with regular and controllable spacing between them, to allow one type of catalytic group to attract and interact with the sulfonic portion of MSA, while the second type of catalytic group attracts and interacts with the methyl portion. This factor can be better understood, if the reader will consider additional comments in PCT application WO 2004/041399, about the symphoric and/or anchimeric traits of a bromate-sulfate reagent that can convert methane into a methyl-bis-sulfate compound.

In view of the foregoing, several potentially important aspects of the MSA-to-ethylene conversion process merit a brief summary, as follows:

(1) A tungsten-treated solid support has been shown to be capable of catalyzing, in a selective and efficient manner (with apparent yields of about 95%), the conversion of MSA (which can be manufactured from “waste” or “stranded” methane) into ethylene, a highly valuable olefin.

(2) The entire series of reactions that lead from MSA to ethylene (presumably via a sulfene intermediate) can be carried out in single reactor vessel. In industry terms, this type of processing is often referred to as “single pot” conversion. By eliminating the requirement for a series of different processing stages (with each such stage requiring its own reactor vessel(s)), costs can be reduced, and yields can be improved.

(3) Solid catalysts having thin-layer surface coatings offer a number of advantages over liquid, gel, “pseudo-liquid” or other catalytic materials that are not well-suited for processing large quantities of liquids or gases.

(4) Because of the commercial, industrial, and manufacturing importance of this discovery, the demonstration of “single pot” processing of MSA to ethylene, using a tungsten catalyst with good yields, should and likely will lead to the evaluation of other candidate catalysts having various similarities to tungsten, including catalysts that contain various transition metals as listed above.

(5) As described in more detail below, catalytic materials that contain some quantity of manganese (which plays a key role in splitting O₂ molecules, in photosynthesis) merit expedited evaluation, and offer good promise for highly efficient catalytic materials for use as described herein.

Various articles and patents contain information that may be able to shed more light on the catalyst-related disclosures herein are listed below, and each listed item also cites additional articles and/or patents, in the footnotes and/or prior art citations. These items merit careful evaluation and consideration, by anyone seeking to fully understand and possibly broaden the disclosures herein and/or the underlying chemical and atomic principles and processes that can enable the types of manufacturing processes disclosed herein. In particular:

(i) Various articles and patents describe the “direct” processing of methane gas on various catalysts. These processes involve contacting methane gas directly with a catalyst, under conditions that cause the methane to be converted into something else. In some cases, the methane is mixed with a second reagent (such as an oxygen-containing reagent) that also contacts the catalyst, so that oxygen atoms will be transferred to the methane, thereby creating an oxygenate (such as methanol, dimethyl ether, or formaldehyde). In other cases, hydrogens are removed from the methane, causing the remaining CH₃ and/or CH₂ groups or radicals to condense into larger hydrocarbons. Pyatnitskii 2003 provides a good review of “direct” catalytic processing of methane, and examples are provided by Wang et al 1995, Pak et al 1998, Makri et al 2003, and U.S. Pat. No. 6,596,912 (Lunsford et al 2003).

(ii) Handzlik et al 2001 describes and illustrates (e.g., in their FIG. 3) complexes and transitional states that may occur when certain types of organic molecules or moieties react with metallic atoms.

(iii) Waters et al 2003 describes transitional states that may be formed when molybdenum or tungsten are used to catalytically dehydrate acetic acid, to form ketene; it also discloses that chromium does not efficiently catalyze that reaction.

(iv) Libby et al 1994 describes the synthesis of C2 through C5 ketenes from their corresponding carboxylic acids, using alkaline (hydroxylated) catalysts.

(v) Wang et al 2004 describes various reactions that tested various catalytic surfaces, which included 100% acidic, 100% basic, 100% redox, mixed redox-acidic, and mixed redox-basic surfaces, using vanadium oxide supports.

(vi) The articles listed under the next subheading, which relate to manganese and photosynthesis, also merit attention.

These are examples of relevant items, and others are also available. However, it must be emphasized and understood that despite very extensive prior work in the field of catalytic processing of hydrocarbons, no one has previously disclosed any system that can enable “single pot” conversion of MSA (which will soon become available in huge quantities, from “waste” methane) into ethylene or other valuable products.

It also should be noted that very little attention has been given, in the prior art, to processing compounds that have a direct bond between a carbon atom and a sulfur atom, as occurs in MSA. Because of the strong electronegativity of sulfur, a direct carbon-sulfur bond (with no oxygen atom between the carbon and sulfur atoms) offers opportunities to manipulate a compound such as MSA in highly useful ways. In the prior art, those opportunities and potentials have received little attention, and little research; instead, in oil and gas processing, sulfur is regarded as a highly unpleasant, unwanted, and toxic pollutant and adversary, which should be eliminated as quickly as possible. That attitude toward sulfur has contributed to a mindset that has prevented the oil and gas industry from recognizing that, under controlled conditions and when using certain specific reagents and pathways, sulfur might provide a perfect “handle” for carrying out highly useful reactions that can provide very high selectivities and yields. The MSA-to-sulfene-to-ethylene pathway offers what appears to be a very good example of that principle and potential.

### Manganese Dopants in Catalysts for Handling Oxygen

Nature (photosynthesis in particular) offers lessons and examples that may well provide a key insight that can help guide and aid the development of highly efficient catalytic materials for use as disclosed herein, especially for reactions that involve the addition of O₂ to a reactor vessel. This category of reactions that involve the addition of O₂ to a reactor vessel includes: (i) the oxidation of SO₂ to SO₃, and (ii) the manufacture of oxygenated compounds, such as methanol, formaldehyde, or dimethyl ether, from methane as a starting point.

Photosynthesis evolved over billions of years in ways that render it remarkably efficient in breaking apart O₂ molecules in ways that allow the resulting “activated” oxygen atoms to be used for assembling larger and more complex molecules. The atomic and subatomic processes involved in these reactions have been given names such as “proton-coupled electron transfer” (PCET), or “hydrogen atom transfer” (HAT), as discussed in articles such as Tommos et al 1998, Westphal et al 2000, and Cukier 2002.

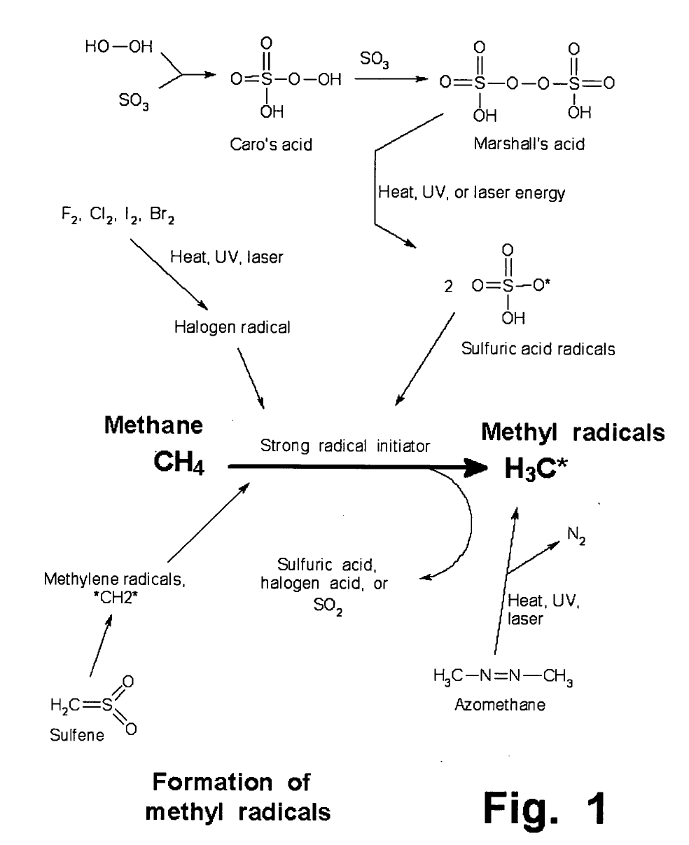

When studying photosynthesis, an important factor to note is that manganese is heavily involved in splitting apart O₂ molecules, to release and “activate” the two oxygen atoms in each molecule of “dioxygen” (O₂). In the chloroplast structures that carry out photosynthesis in plants, manganese atoms are grouped together into “tetra-manganese clusters”, with each cluster containing four manganese atoms connected to each other by “bridges” formed by oxygen atoms. These tetra-manganese clusters are illustrated in FIG. 4 of Tommos et al 1998, FIG. 1 of Westphal et al 2000, and FIG. 1 of Cukier 2002.

This patent application is not an appropriate place for a more detailed analysis of how manganese helps plants carry out photosynthesis, since that information is complex and is already available in various articles, including the three articles cited above. However, it is noted and disclosed herein that solid-supported catalysts that contain manganese and oxygen (and possibly other elements, such as tungsten, molybdenum, etc.) are likely to be able to emulate the highly efficient mechanisms of photosynthesis, in ways that can be adapted to increase the rates and yields of chemical processing as disclosed herein.

It also is disclosed herein that solid-supported catalytic surfaces containing manganese (and possibly other catalytic metals, such as tungsten, molybdenum, etc.) are likely to offer substantial improvements in photovoltaic materials that can convert sunlight or other radiation into electrical voltage and current. This is a separate field of research that merits and needs attention in its own right. Even though photovoltaic materials do not directly relate to the chemical processing of hydrocarbons as disclosed herein, the insights and computer modeling that have been performed to date on candidate catalysts for performing certain types of oxygen activations and transfers, in the realm of chemical processing discussed herein, may well have laid the groundwork for developing similar advanced catalytic materials derived from these disclosures, which appear likely to enable more efficient photovoltaic generation of electricity, from sunlight, than has ever been previously possible under the prior art.

### “Outer” Anhydrides

Analysis of another article (Karger and Mazur 1971, entitled, “Mixed sulfonic-carboxylic anhydrides: I. Synthesis and thermal stability. New synthesis of sulfonic anhydrides”) suggested to the Applicant herein that certain additional processes might also be involved (or might be created, by controlling reaction conditions) in the formation of sulfene from MSA, and in subsequent polymerization reactions involving the sulfene.

Karger and Mazur worked with MSA (which is listed as their formula CH₃SO₃H a number of times, such as in their Tables I and II on page 530); however, they were combining MSA with various acid chloride compounds, in ways that displaced the chloride moieties and created various ether and/or ester linkages in the resulting anhydrides. One passage on page 531 is worth particular attention. It reports, “Thus, methanesulfonic anhydrides decomposed only above 250° [it should be noted that the quoted phrase is ambiguous; it may refer to only those anhydrides that would decompose only above the 250° C. temperature, or it may refer to all anhydrides, if treated at temperatures above 250° C.] to give methanesulfonic acid (70%), residual intractable polymer (15%), and sulfene which presumably did not survive its conditions of generation (equation 8).” Their equation 8 was:

CH₃SO₂OSO₂CH₃--->CH₃SO₃H+(CH₂═SO₂)

at 250° C.

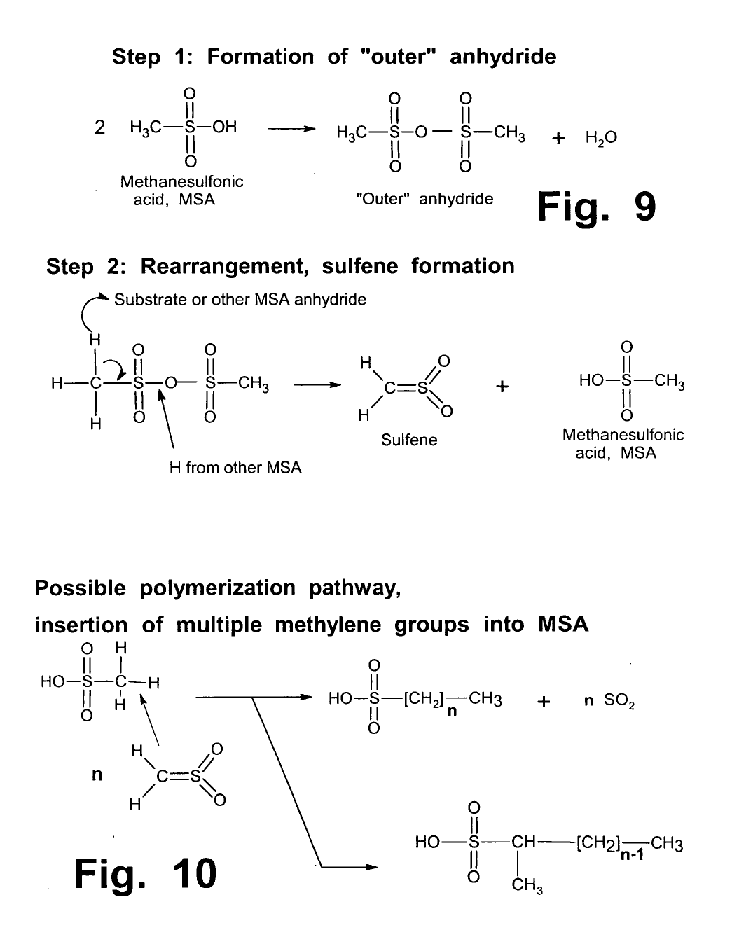

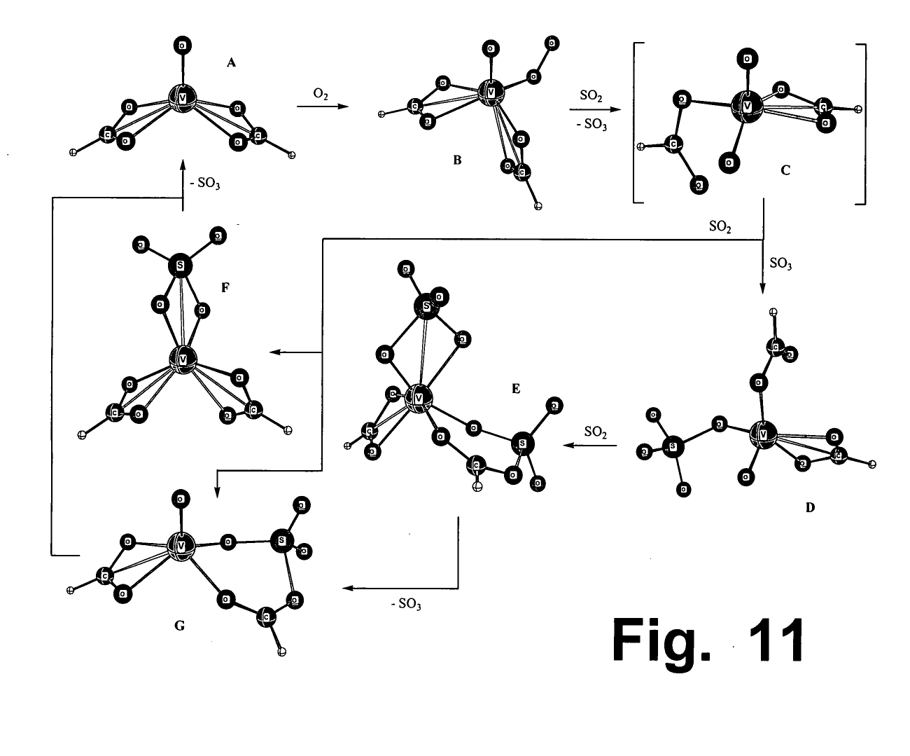

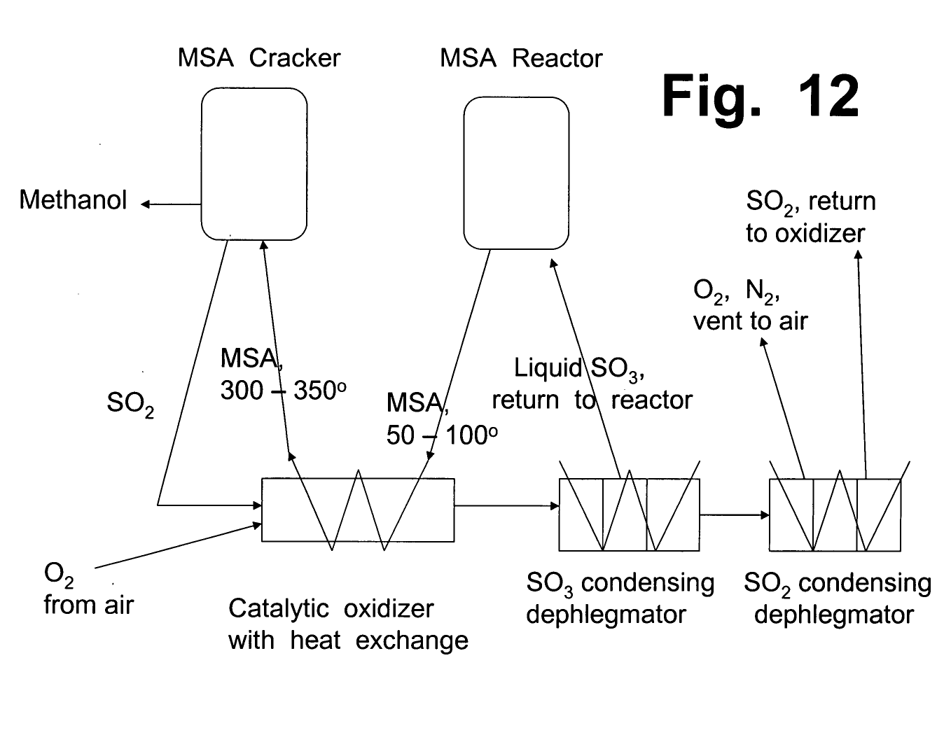

Two reaction pathways that offer candidate mechanisms for explaining the formation and then destruction of the MSA “outer anhydride” are shown in FIG. 9. The first reaction in FIG. 9 shows a condensation step involving two molecules of MSA, which creates an “outer anhydride” of MSA (shown as the starting reagent in Karger's Equation 8) while releasing a molecule of water. To create this condensate, the sulfate group on a first molecule of MSA releases a hydrogen proton, and the sulfate group on a second molecule of MSA releases a hydroxy group. These two reactions join the MSA residues together through a single-bonded oxygen linkage, which can be regarded as an ether bond (or thioether bond, since the oxygen atom links two sulfur atoms), or as an ester (or thioester) bond, since the sulfur atoms also have double-bonded oxygen atoms. Presumably, this reaction can be promoted by dehydrating agents such as mentioned elsewhere herein, and in various passages in Karger et al 1971.

In Step 2 in FIG. 9, which likely will occur only at relatively high temperatures, the “outer anhydride” molecule rearranges. This reaction is postulated to involve: (i) release of a hydrogen proton from the methyl group of the anhydride; (ii) migration of the electrons from the C—H bond over to the C—S bond, thereby forming a double bond; and, (iii) breakage of the S—O linkage, in the presence of protons in the acidic MSA solution. It is possible but not especially likely that the same hydrogen proton from a particular molecule will bond to the oxygen atom from an S—O linkage that is being broken in that same molecule. In addition to generating sulfene, as shown in FIG. 8, the rearrangement in step 2 also regenerates and releases a molecule of MSA.

It is also worth noting that because of how the “outer anhydride” breaks apart, it might be useful as a radical initiator compound, to trigger the conversion of methane into methyl radicals, during the processing that converts methane into MSA as shown in FIG. 2 herein. If the “outer anhydride” compound is broken apart by means such as passing it across a heating element in a device such as “radical gun”, it may release at least one and possibly two “strong radical initiator” compounds that can efficiently remove hydrogen atoms from methane. This possibility is especially interesting because its products may be able to reform MSA, rather than creating a sulfuric acid waste, which will be created if Marshall's acid is used.

Additional comments on sulfene formation, and on Karger et al 1971, are provided in King and Rathore 1991. Those comments, while focusing on different aspects of the chemistry (such as IR spectroscopy of sulfene at low temperatures) are nevertheless believed to be consistent with all disclosures and postulated mechanisms herein.

### Polymerization and Dendrimers

As mentioned above, one comment made in passing by Karger et al 1971 reported, on page 531, “a black intractable polymeric solid, which gave no acid reaction on boiling with water, was the only nonvolatile product”. The yield of Karger's polymer was low (15%), their compound was never analyzed, and it clearly did not teach or suggest any practical way to manufacture a commercially viable polymer. Their publication occurred more than 30 years ago, and it never led to any commercialization.

However, the discoveries and disclosures of the Applicant herein appear to be approaching a point where practical and efficient methods can now be disclosed for manufacturing various types of polymers from methane, via MSA and MSA anhydrides. In particular, the Applicant herein believes and anticipates that polymeric material can be created by repeated insertions of methylene groups (—CH₂—) into growing carbon chains, as indicated in FIG. 10. It is possible that methylene groups can be inserted into the carbon-sulfur bond, shown in the MSA molecule that serves as the starting point for the chain-lengthening reaction; however, computer modeling indicates that the more likely point of insertion appears to be at a carbon-hydrogen bond in the methyl group.

Regardless of which bond provides the particular insertion site, a hard polymeric compound was indeed observed, when the “outer anhydride” of MSA (purchased in crystalline form, from Aldrich Chemicals) was heated to a temperature higher than 250° C., under nitrogen gas. The decomposition created both a clear liquid, and a black solid. Both the liquid and the residue were chemically analyzed. The clear liquid was found to consist mainly of MSA and cycloalkanes. The black solid was found to contain cyclic hydrocarbons, naphthenics, and a relatively high quantity of aromatic structures. Some of the aromatic rings were bridged by sulfonate or methylene bridges, and some of the aromatic rings had cyclopropane rings attached to them.

Based on those results combined with other teachings herein, it is believed and anticipated that practical means for making commercial quantities of hydrocarbon liquids that can be used as fuels, and possibly as chemical feedstocks, can now be identified and developed, using pathways that pass through MSA, MSA esters, and/or MSA anhydrides, by using steps that include the following:

a. creating a preparation that contains sulfene and/or MSA outer anhydride, mixed with a suitable solvent having a boiling point higher than the decomposition temperature of the sulfene or sulfene-containing starting material (dimethyl sulfoxide offers one candidate for early evaluation, and other solvents with higher boiling points are known); and,

b. heating the preparation, while bubbling nitrogen gas up through it at rates that will remove the desired products as they are being formed, without allowing them to continue to rearrange until they form aromatic rings.

In addition, it is believed and anticipated that practical means for making commercial quantities of solid polymers can now be identified and developed, using pathways that pass through MSA and MSA anhydrides, by using steps that include the following:

a. creating a preparation that contains (i) sulfene and/or MSA outer anhydride, and (ii) any other desired starting reagent (such as a styrene precursor, acrylate precursor, vinyl precursor, etc.), in a suitable solvent having a boiling point higher than the decomposition temperature of the starting mixture; and,

b. subjecting the preparation to a “cooking” reaction (i.e., involving a controlled temperature-pressure-time combination) that forms a desired solid, while bubbling an inert gas (such as nitrogen or CO₂) through the mixture at rates that are sufficient to remove the desired products before they form aromatic rings.

In either type of system, the use of solvents with boiling points that are well above the heating temperatures being used, combined with the use of gas “sweep” systems to promptly remove desired products in gaseous phase as they are formed, provides a useful and flexible means for controlling the reactions.

On the subject of reactions that can create hydrocarbon chains by insertion of methylene groups, a report by Michalak and Ziegler 2003 should also be noted. This report indicates that branched polymers can be created, in controllable manners, by using certain types of catalysts, such as nickel-diimine or palladium diimine.

This approach to controlling the branching of hydrocarbon chains that are being formed has a number of important commercial implications. One application worth noting relates to the manufacture of liquid fuels having higher energy density per volume, as well as higher quality (including higher “octane” ratings, for gasoline). These aspects, involving increased value and utility, arise from two facts. First, in a hydrocarbon liquid, molecules that have some degree of branching tend to fit together better (allowing greater weight per volume) than entirely linear molecules. Second, molecules that have some degree of branching are not as long as straight-chain molecules, for a given number of carbon atoms, and there is less chance that the “far end” of some particular molecule will be pushed away, in unburned form, when one of the molecules goes through rapid and explosive but imperfect combustion. As an illustration of this phenomenon, the molecule with the gold-standard “100” octane rating is 2,2,4-trimethyl pentane, rather than straight-chain octane.

Other potential applications (including the manufacture of various types of plastics and polymers, including isotactic, atactic, or other “designed” polymers) will be recognized by those skilled in the art, after they have studied the properties and potentials of sulfene as a methylene transfer agent.

Two additional classes of reactions also should be noted, involving C═O double bonds, generally referred to as carbonyl bonds or groups. If sulfene transfers a methylene group into an aldehyde group (i.e., a carbonyl group located at the end of a carbon chain) or into a ketone group (i.e., a carbonyl group located in the middle of a carbon chain), the insertion will create a three-membered oxirane or epoxide ring, which will include the carbon atom that had the carbonyl group. Epoxide and oxirane rings are unstable and reactive, due to their stressed bond angles. This makes them useful reactants in certain types of chemical processing, if they can be used rapidly after they are generated, before they have time to spontaneously decompose.

It should also be noted that sulfene may become useful in modifying the surfaces of various types of silicate materials that will have special properties or uses following such treatments. Examples of such candidate uses include semiconductors, and an emerging category of materials that are creating new types of interfaces and interactions between biological materials (such as antibody fragments or other proteins, DNA segments, etc.) and nonbiological materials, for purposes such as diagnostic, therapeutic, or other analytical, processing, medical, or other physico-chemical uses. Anyone interested in this category of uses should study Lie et al 2002, including passages such as the first full paragraph on page 116, which discusses the formation of direct silicon-carbon bonds rather than silicon-oxygen-carbon linkages, and the last paragraph in column 1 of page 117, which discusses the possible insertion of methylene groups (—CH₂—) into silicon-silicon bonds.

### “Upstream” Options and Enhancements

In addition to the discussion of synthesis and use of MSA and its esters and anhydrides, in the foregoing sections, this application also contains a number of teachings on other aspects of the overall system. As described near the start of the “Detailed Discussion” section, these disclosures are intended to help ensure that any and all “disclosure of the best mode” requirements for valid patents are satisfied, since they relate to improved ways for designing and operating complete and functional systems that can take methane gas all the way to liquid fuels, olefins, polymers, and other valuable compounds.

The disclosures in this subsection relate to “upstream” processing, i.e., steps that help promote the synthesis of MSA, the crucial intermediate, from methane. These “upstream” options and enhancements include the following:

(1) It is believed and anticipated that if carbon dioxide (CO₂) is pressurized to a point that causes it to become a supercritical liquid, it may be able to increase the solubility of methane gas, in a liquid solution of SO₃ and MSA. If this is confirmed in continuous-flow testing, the use of supercritical liquid CO₂ may be able to increase and improve the mass transfer rates that will transfer gaseous methane into a liquid solution. This may be able to increase the speed and efficiency of the reaction that converts methane into MSA.

(2) It is believed and anticipated that it may be possible to adapt either the inner anhydride of MSA (i.e., sulfene), or the outer anhydride of MSA, for use as a radical initiator compound, instead of Marshall's acid or various other radical initiators that will generate acidic waste byproducts. Briefly, a radical initiator compound that can efficiently remove a hydrogen atom (both a proton, and an electron) from methane, thereby converting the methane into a methyl radical, H₃C*, is necessary to launch the chain reaction that will convert methane into MSA, as shown in FIG. 2. As described above, sulfene can release methylene radicals. These radicals can be regarded as “double-strong” radicals, since they have not just one, but two unpaired electrons.

When a methylene radical (with two unpaired electrons) reacts with methane, the “double-strong” methylene radical is likely to remove a single hydrogen atom from methane. This will balance out the two molecules, making them equal, thereby creating two methyl radicals, H₃C*. Each of these methyl radicals will be able to combine with sulfur trioxide, SO₃, to form MSA radicals, as shown in FIG. 2, and the MSA radicals will then remove hydrogens from fresh methane, to form stable MSA while creating new methyl radicals that will keep the chain reaction going.

Accordingly, if sulfene (in gaseous, mist, or similar form) can be injected into a methane stream, it may be an effective and useful radical initiator compound, which may eliminate or reduce the need for Marshall's acid, halogen gases, or other compounds that would likely create acidic wastes.

If desired, an MSA anhydride can be pumped out of a device (which can be called a “radical gun”) having a nozzle that contains a very hot electric filament or other heating element (which can be embedded in a quartz tube or other protective device, if desired) that will break apart the radical-releasing molecules as they pass across the heating element. These types of devices are described in numerous articles, including Danon et al 1987, Peng et al 1992, Chuang et al 1999, Romm et al 2001, Schwarz-Selinger et al 2001, Blavins et al 2001, and Zhai et al 2004. Similar devices can be constructed and tested, which will pass a selected radical-releasing compound through a nozzle or other component having a zone that is subjected to high levels of ultraviolet, tuned laser, or other radiation (or, indeed, any other form of energy input).

(3) One or more types of borate compounds (such as trimethyl borate, or borate anhydride), if properly utilized in the MSA reactor vessel, may be able to help promote the synthesis of MSA, mainly by reducing unwanted SO₃ reactions (such as the formation of CHₓ(SO_{y})ₙH polymers and other species, where x, y, and n are variables). In addition to helping to minimize and prevent the formation of unwanted methyl-sulfonate species, the borate compound can also help maintain SO₃ molecules in their alpha and gamma forms, which can help improve the overall conversion of SO₃ to MSA. Such borate compounds can be coated onto immobilized or particulate surfaces, to ensure that they remain inside the MSA reactor.

(4) If quantities of both liquid and gaseous SO₃ are pumped into the MSA reactor vessel (either separately, through different inlet nozzles, or in a mixed and entrained stream, or in any other suitable manner), the mixed liquid and gas streams may be able to react with methane gas, in the liquid/gas mixtures and interfaces that will be present inside the reactor, in ways that will increase the rates of MSA formation.

(4) It may be possible to use methods for breaking apart a radical initiator (such as Marshall's acid, or DMSP as disclosed below) using methods such as photolysis, in ways that create or preserve certain types of electron “spin” in the two radicals that are formed when the radical initiator breaks apart. This is analogous to creating one radical with an electron having a “right-handed” spin, while the other radical has an electron with a “left-handed” spin. This can be important, because two right-handed radicals cannot recombine with each other, and two left-handed radicals cannot recombine with each other, in ways that would reform the initiator and “quench” it as a radical. In other words, a left-handed spinning radical must combine with a right-handed spinning radical, to recombine. This approach suggests useful methods (such as continuing to shine light having a radical-breaking wavelength) into an MSA reactor vessel), through one or more transparent panels in one or more walls of the reactor.

By contrast, breakage of a radical initiator by means such as heating provides less of this useful effect, and allows radicals to recombine more readily.

These factors are discussed in more detail under the term “solvent cage” effects, in various chemical articles. If those factors are recognized and understood, they can be put to good use in the systems disclosed herein.

(5) It may be possible to create MSA, in bulk quantities, by using one or more types of surface-active radical initiators, comparable to the immobilized catalytic compounds described by Barteau 1996 (for the formation of ketene, as described in the Background section) or described herein, in the passages on candidate pathways for making sulfene. This approach is supported by those teachings, combined with the additional teachings of Lie et al 2002, which is entitled, “Photochemical reaction of diazomethane with hydrogen-terminated silicon surfaces”, describing work done in the laboratories of Benjamin Horrocks and Andrew Holton, at the University of Newcastle upon Tyne, in Great Britain.

The Lie et al 2002 article describes highly complex and sophisticated chemistry that was done using light-activated molecules on silicon surfaces. Thus type of photo-catalyzed chemistry is used to create the extraordinarily tiny circuitry in integrated circuits, and there is no reason to suspect or assume that this class of chemistry could be adapted and converted into efficient methods for mass-manufacture of liquid chemicals, at the scales involved in methane conversion.

Nevertheless, various factors discussed herein led the Applicant to carefully study various articles on chemical treatment of semiconductor surfaces (such as Barteau 1996 and Lie et al 2002), and those articles triggered several insights by the Applicant, into ways that certain chemical reactions and pathways used in preparing semiconductor surfaces might be adapted and expanded to enable the handling and processing of bulk liquids, such as methane, MSA, and sulfene.

In particular, certain passages in Lie et al 2002 (especially a passage that begins with the first full paragraph in column 2 of page 113) state or imply that certain types of radical species can be generated, at or near the surfaces of silicon-containing materials, when compounds such as diazomethane are activated by using certain conditions (mainly involving ultraviolet light radiation, when semiconductor manufacturing is involved). Those passages, combined with additional teachings in Barteau 1996 and other articles, have suggested to the Applicant that if certain types of solid materials are surface-treated in certain ways, the resulting surface-treated supports may be able to function as efficient removers of hydrogen atoms (both protons and electrons) from lower alkyl molecules such as methane, or from other compounds (such as azomethane, sulfene, ketene, etc.) that can subsequently function as “strong radical initiators” (i.e., compounds that can efficiently remove hydrogen atoms from methane or other lower alkanes). This would generate methyl radicals, in quantities that may be able to initiate the methane-to-MSA conversion reaction shown in FIG. 2, without requiring a slow and steady input of radicals from a radical initiator compound such as Marshall's acid or a halogen gas.

Accordingly, this approach offers a promising candidate pathway for use as disclosed herein. Those skilled in this field of art can better understand and evaluate these comments if they study Lie et al 2002, Barteau 1996, and other published works cited by those authors, especially including the items cited as footnotes 22, 33-34, and 41-52 by Lie et al. Another “upstream” option involves the use of DMSP as a radical initiator. Because of its potential importance, it is discussed under a separate heading.

### Use of DMSP as a Radical Initiator

The reaction that causes methane to bond to SO₃, forming MSA as shown in FIGS. 2 and 3, requires the use of small quantities of a “radical initiator” that will initiate a chain reaction.

Although a fairly wide range and assortment of candidate radical initiators are illustrated in FIG. 1, none of them are ideal, since they all create unwanted byproducts. For example, if Marshall's acid (shown at the top of FIG. 1) is used, its byproduct will be sulfuric acid. Even though that byproduct will be created only in quantities that are small and limited, compared to the very large outputs of MSA, the creation of unwanted sulfuric acid can pose costly and potentially severe problems, when scaled up to the huge quantities of methane that can be converted into easily-transported liquids by this process.

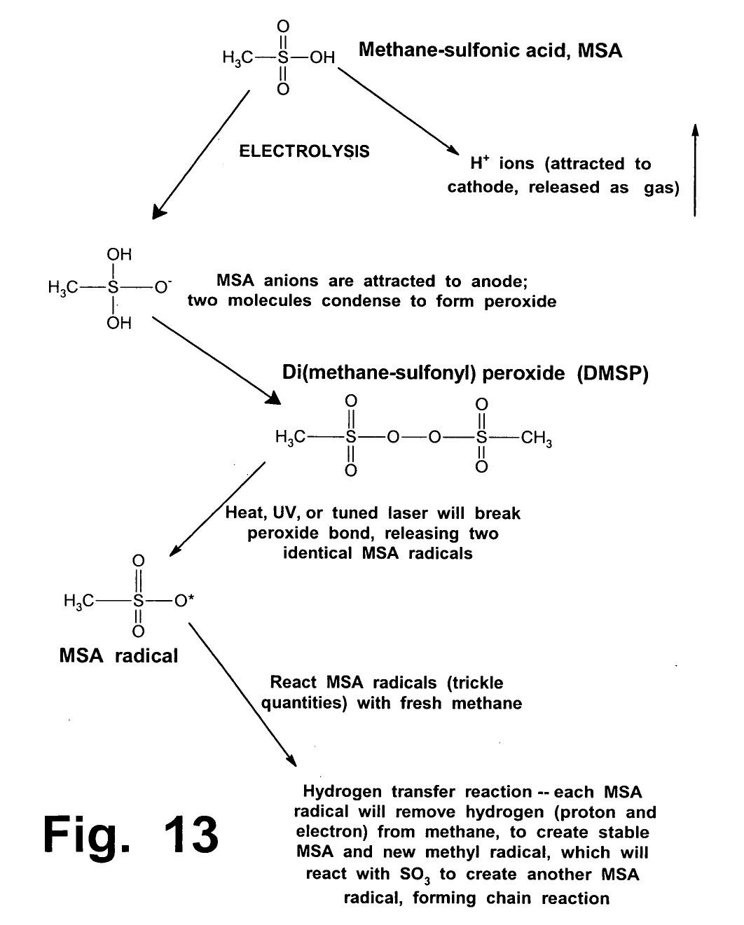

Accordingly, it is disclosed herein that a compound called di(methane-sulfonyl) peroxide (abbreviated as DMSP, with the formula H₃CSO₂O—OSO₂CH₃, as shown in FIG. 13) offers another candidate radical initiator.

DMSA can be prepared directly from MSA, as a condensate (or dimer), by using electrolysis. To carry out this process, two electrodes are submerged in a liquid solution of MSA. Unless and until testing with various candidate solvents or other additives (which may be able to promote and facilitate the process) indicates otherwise, a presumption arises that the MSA solution preferably should be as pure as possible. The supply of MSA for the electrolysis can be provided from any available source. When a plant is just getting started, the MSA can be delivered in containers, from an outside source; subsequently, after the plant is running, the MSA can be obtained as a small portion of the output from an MSA-forming reactor vessel.

A strong electrical voltage is imposed across the two electrodes that have been submerged in the MSA. This voltage will cause the cathode to have a negative charge, which will attract positively-charged cations; the cathode will supply electrons to those cations, in a process called reduction. The anode will have a positive charge, which will attract negatively-charged anions.

The electrodes used in electrolysis can have any desired shapes. In laboratory settings, they often are cylindrical rod-like devices, which can simply be lowered into a beaker and held in position by a clamp. In industrial operations, they often are shaped as flat parallel plates, which often are provided as a series of multiple flat parallel plates having alternating positive and negative charges.

Since MSA is an acid, some of the molecules in the acid will naturally and spontaneously dissociate, in a way that releases H⁺ cations and H₃CSO₃⁻ anions. When a strong voltage is imposed on liquid. MSA, by electrodes submerged in the liquid, the H⁺ cations will be attracted to the negatively-charged cathode, while the H₃CSO₃⁻ anions will be attracted to the positively-charged anode. To some extent, passage of a voltage through MSA may increase the rates of ionic dissociation of the acid.

As H⁺ cations in the liquid gather around the cathode, they are provided with electrons, by the electrical current that is being pushed into the liquid by the cathode. The electrons that emerge from the anode will initially convert the H⁺ cations into radicals, designated herein as H*, where the asterisk indicates an unpaired electron. These radicals are highly unstable, and two H* radicals will bond to each other. This creates hydrogen gas, H₂, which creates bubbles on the surface of the cathode. These bubbles will grow and enlarge, until their buoyancy causes them to break away from the cathode surface and float to the surface of the liquid. Whenever hydrogen gas is formed by industrial or other large electrolysis units, gas collectors must be used, because hydrogen gas is explosive, and it must be handled safely.

At the same time, H₃CSO₃⁻ anions will be gathering around the positively-charged anode surfaces. These MSA anions will surrender an electron to the anode (thereby completing a circuit, and establishing an electrical current through the liquid, driven by the voltage that is being imposed on the electrodes and the liquid).

When an MSA anion loses an electron, it becomes an MSA radical, as shown in FIG. 13. Since the unpaired electron is on one of the oxygen atoms that is bonded to the sulfur atom, these MSA radicals also can be referred to as methyl-sulfonyl-oxyl radicals.

Two such radicals will bond to each other, forming a peroxide linkage at the center of a condensate (also called a “dimer”, since it is formed from two units). That condensate is DMSP, as described above and shown in FIG. 13. It is, in effect, an analog or variant of Marshall's acid, with two dimethyl groups added (symmetrically) to the two ends of Marshall's acid. The presence of the two methyl groups helps stabilize DMSP, making it easier to store, transport, and use DMSP, compared to Marshall's acid.

When the time arrives to use DMSP as a radical initiator, to start a chain reaction that will combine methane with SO₃ to form MSA, the DMSP reagent can be treated by a suitable energy input (such as mild heating, ultraviolet radiation, or a tuned laser beam), in a way that will break the peroxide bond in the center of the DMSP molecule. This will release two identical radicals, which are radical forms of MSA, having the formula H₃CSO₂O*. Since these radical are highly unstable, they should be created immediately before use, such as by passing them through a heating, UV, laser, or similar radical-creating device (which can be referred to by terms such as radical gun, radical nozzle, radical injector, etc.) which is affixed directly to a side or end of the MSA-forming reactor vessel. If desired, a plurality of radical nozzles can be distributed around the methane inlet of such a reactor.

For reasons that arise from a subatomic process called “electron spin”, it is believed that the use of UV or tuned laser radiation is likely to be preferable to heating, as an energy input that can drive the cleavage of DMSP to release MSA radicals. Accordingly, suitable radical injection means can be provided by, for example, passing DMSP in liquid form through a tubing segment that has transparent walls (which can be made from various known types of specialized glass, quartz, carbonate, or other corrosion-resistant materials). This will allow the DMSP molecules, as they pass through the transparent piping segment, to be exposed to focused a source of UV radiation, or to a laser source that has been “tuned” to a particular frequency that has been optimized for breaking apart the peroxide bond in DMSP. The transparent tubing segment that provides the radical injector device can have any desired cross-sectional shape, size, and other features. For example, to increase the efficiency of the DMSP cleaving process, DMSP can be passed through a wide and relatively thin rectangular segment. This segment can have a glass or other transparent face on one side, to allow entry of the UV or laser radiation into the DMSP liquid, and it can have a reflective mirror-type surface on the opposing side, to reflect any radiation that was not absorbed during its “first pass” through the liquid, back into the liquid, for additional “second pass” absorption.

The MSA radicals that will be released, when DMSP is cleaved in this manner, will react with fresh methane that is being pumped into the reactor vessel. This reaction will efficiently remove a hydrogen atom (both proton and electron) from the methane, and transfer that electron to the MSA radical. When that hydrogen transfer occurs, it will create two compounds:

(1) stable and complete MSA, which is the desired product of the reaction; and,

(2) new methyl radicals, which will keep the chain reaction going. Since methyl radicals are not strong enough to take anything away from SO₃ molecules, they will cling and bond to SO₃ that is being pumped into the reactor. That reaction will form new MSA radicals, which will then react with still more fresh methane, continuing the chain reaction.

Accordingly, DMSP, which can be formed as a peroxide dimer by electrolysis of MSA, appears to offer an optimal radical initiator for enabling MSA production. The DMSP initiator can be manufactured inexpensively, using a small fraction of the MSA being created by the MSA-forming reactor, and it will not create any substantial quantities of sulfuric acid or other unwanted byproducts.

It should also be noted that a new composition of matter is disclosed herein, comprising the reaction mixture that will be contained within the reactor vessel that is being used to manufacture MSA. This composition of matter contains a mixture of methane, methyl radicals, SO₃, MSA, and MSA radicals, further characterized by the absence of any significant quantity of any unwanted byproduct(s) that would be created by a radical initiator other than DMSP and/or MSA radicals. Such an unwanted byproduct is exemplified by sulfuric acid, which is created when Marshall's acid is used as a radical initiator. This reaction mixture is also characterized by another limitation: the components of the reaction mixture (i.e., methane, methyl radicals, SO₃, MSA, and MSA radicals) must be present in concentrations that will enable the mixture to sustain an ongoing chemical chain reaction, which allows MSA to be continually removed from the reactor vessel while fresh methane and fresh SO₃ are pumped into the reactor vessel.

### Manufacture and Use of Dimethyl Ether (DME) from MSA

Various methods are known in the prior art for making a fuel called dimethyl ether (abbreviated as DME, with the formula H₃COCH₃). For example, DME can be made by using a dehydrating agent (such as zinc chloride) to directly remove a water molecule when two molecules of methanol are condensed, as described in, e.g., U.S. Pat. No. 2,492,984 (Grosse & Snyder 1950). DME also can be made by passing methanol through a suitable Zeolite-type of material (e.g., U.S. Pat. No. 3,036,134, Mattox 1962). It also appears likely to be possible to convert MSA directly into DME, by passing the MSA through a suitable Zeolite material, based on comments in items such as U.S. Pat. No. 4,373,109 (Olah 1983), Olah 1987, and Zhou et al 2003.

Recently, DME has become of interest as a fuel or fuel additive that is ideally suited for a number of uses, because of a combination of factors. It is less corrosive than methanol, and can be shipped and stored in pipelines, tanks, or other vessels made of conventional steel, without requiring special precautions. It will readily convert between a liquid and a gas, at moderate operating pressures that can be achieved by inexpensive tanks, and it burns quickly, cleanly, and thoroughly, without creating any soot, smoke, odors, or other residues, and without posing a risk of carbon monoxide poisoning in homes that are not adequately ventilated. Because of these properties, DME is widely used in many rural and less-developed parts of the world as a “bottled gas”, for uses such as indoor cooking. When used for such purposes, it can utilize the same types of tanks, valves, and burners that are used to store and burn “liquified petroleum gas” (LPG), which mainly contains propane and butane.

In addition, DME has enough energy content to be well-suited for use in diesel engines and in turbines, and it also can be used as a propellant, for pressurized cans that hold aerosol sprays, as a substitute for chlorofluorocarbons (CFC's), which are environmentally dangerous. More information on those uses is available from the International DME Association (IDA, www.vs.ag/ida), and from websites such as www.aboutdme.org and wwwjfe-holdings.co.jp/en/dme.

It is also disclosed herein that DME appears to be well-suited for use in supplementing methane, in natural gas pipelines that serve homes and factories, in a process that is comparable to using propane-air mixtures, for an operation that is usually called “peak shaving” by people who work at gas utilities. This option is described in more detail below. When DME is used for this purpose, it will not require any adjustments to burners or other devices, so long as the mixture of DME and a second gas (such as air, nitrogen, etc.) is adjusted to approximate the “Wobbe index” of the gas that is normally distributed by a particular pipeline system.

It should also be recognized that, since DME is effectively a condensed version of methanol (with water removed), its production at oil or gas producing sites around the world can provide two potentially enormous benefits. First, if water is released (such as in the form of steam) at an oil or gas production site, the steam can be condensed into readily drinkable (potable) water which is suited for drinking, domestic uses, irrigation or livestock purposes, etc. This can be extraordinarily valuable in numerous countries with large oil and/or gas reserves but without sufficient fresh water (such as Saudi Arabia and Algeria, as just two examples).

The second benefit arises from substantially reduced transportation costs, which can be provided by removing water from the cargo at the source location, instead of paying to ship that water across an ocean or through a pipeline. Even if the ultimate goal is to get methanol to a destination point, it can be more economic to remove the water from the methanol at the source location, ship “dehydrated methanol” (in the form of DME) via a tanker or pipeline, and then add water back to the DME (to reconstitute it into methanol) after it reaches the destination. Similar processes have been used for decades to minimize the costs of storing and shipping various types of semi-dewatered products, such as condensed fruit juices.

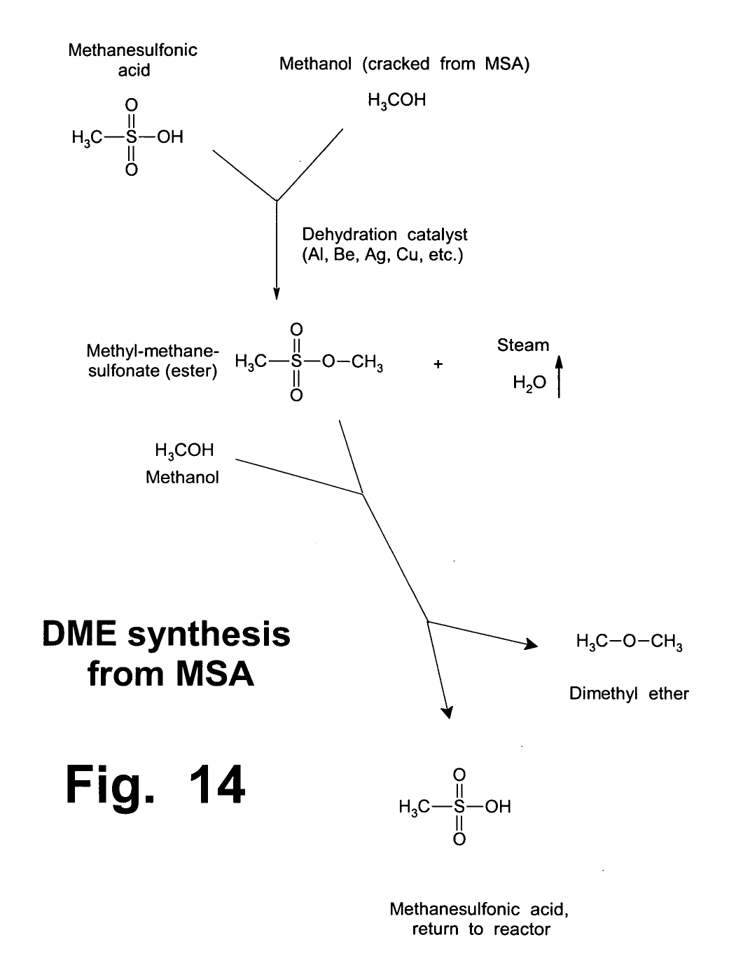

Accordingly, an alternate route for manufacturing DME from MSA is disclosed herein, and is illustrated in FIG. 14. The initial reaction, shown at the top, involves combining MSA with methanol. As indicated in PCT application WO 2004/041399, this methanol can be created by “cracking” MSA at a suitable temperature and pressure, in the presence of a suitable catalyst. Accordingly, MSA is the only feedstock needed for the overall process, if a portion of the MSA stream is diverted to a cracking unit and used to provide the methanol.

When MSA and methanol are combined under a suitable pressure-temperature combination in the presence of a dehydrating catalyst (candidate metals that merit evaluation for such use include aluminum, beryllium, silver, copper, etc.), the two compounds will form at least one condensation product, such as methyl-methanesulfonate, abbreviated as MMS and having the formula H₃CSO₃CH₃. Because of the arrangement of the oxygen atoms around the sulfur atom in MMS, as shown in FIG. 14, this compound can be regarded as an ester (or a thioester, since it includes sulfur). Water, in the form of steam, will be released when MMS is formed.

In the next step of the reaction, additional methanol is added to MMS. DME will be formed, as the desired product, while MSA also will be released. The MSA can be returned to the top of the reaction pathway; alternately, since it will be at an elevated temperature, it can be sent to the cracking unit, to release more methanol from it, with minimal heating costs.

Either or both reactions in this pathway can be carried out in a reactor that can be designed and operated as a “reactive distillation column”, using methods known to those skilled in the art. DME will be one of the lighter products, and it can be withdrawn in the distillate fraction.

In some respects, this pathway is analogous to a different pathway disclosed in U.S. Pat. No. 6,518,465 (Hoyme et al 2003), which converted an alkyl ester (such as methyl acetate) into a carboxylic acid (such as acetic acid). An ether compound such as dimethyl ether was formed as a byproduct of that pathway. However, a complete reading of that patent indicates that DME was not regarded as the main product; indeed, the patent teaches that any DME formed by that process can be hydrolyzed, to convert it into methanol. That was entirely logical, based on what Hoyme et al were trying to accomplish; since methanol is a stable liquid (while DME wants to become a gas, and requires constant pressure to prevent it from doing so), methanol generally is easier and safer to handle, in the types of settings contemplated by Hoyme.

### Use of DME to Supplement Natural Gas in Pipelines

It is also disclosed that DME apparently can be used to make up shortages of natural gas that is being distributed to homes and factories, via pipelines. In numerous regions of the world, the threats of natural gas shortages are becoming acute, in light of factors such as disruptions to oil and gas production in offshore and coastal regions due to hurricanes and typhoons, the growth of energy consumption in nations such as China and India, which have caused greater competition for energy supplies around the world, terrorist attacks and political instability, and explosions or other accidents involving aging equipment and other problems.

Driven by these and other factors, a method for using propane to supplement natural gas supplies has been developed. This process is often called “peak shaving”, since it was developed to help smooth out the peaks in demand that occur in many distribution systems during certain hours. Machines and methods for creating propane-air mixtures, and for injecting those mixtures in gas supplies being carried by pipelines, have been developed and are described elsewhere. For example, a company called Standby Systems, Inc. (Minneapolis, Minn.) has posted, on its website, extensive information on its systems and services, including an extensive summary (with illustrations and data tables) that can be obtained at www.standby.com/propane/pdf/pss_ovw_e2e.pdf.

One of the crucial measurements that enables pipeline companies to smoothly and efficiently combine controllable propane-air mixtures with natural gas supplies, without requiring any adjustments to burners or other devices in factories or homes, is called the “Wobbe index”. This number is calculated, first, by determining the “higher heating value” of a fuel gas. The reference to “higher” heating value (also called gross heating value) assumes that water vapor in exhaust gases is condensed back to liquid; “lower” Wobbe index numbers also can be calculated if desired. That heating value number is then divided by the square root of a fuel gas's specific gravity (the specific gravity is the ratio of a gas's molecular weight, to the molecular weight of air).

Accordingly, a Wobbe number is expressed in terms of heating value, per volume of gas (ignoring the fact that the volume number is actually a square root of a density). If the work output number is measured in terms such as kilocalories, the resulting numbers are greater than 10,000. To avoid those awkward numbers, the commonly used system uses “megajoules” (abbreviated as MJ) as the number for measuring the heating value of a fuel gas. Typical Wobbe index numbers for most fuels of interest range from about 40 to about 80; for example, the Wobbe index for pure methane (with one carbon atom) is 53.454, while the Wobbe index for pure propane (with three carbon atoms) is 81.181.

More information on Wobbe index numbers and calculations is readily available from an Internet search, and from various articles and patents, such as U.S. Pat. No. 6,896,707 (O'Rear et al 2005), entitled, “Methods of adjusting the Wobbe Index of a fuel and compositions thereof”.

Natural gas that runs through pipelines can vary substantially in its energy content and/or specific gravity, depending on the concentrations of non-methane components. For example, ethane and propane have higher energy content, so they will make a gas supply “richer”. Nitrogen and carbon dioxide are inert, and will make a gas supply “leaner”. Each gas supplier knows the Wobbe index of the gas it is pumping into its pipelines on any given day; therefore, if its gas supply must be supplemented by a propane-air mixture, the pipeline company will adjust the propane-air mixture to closely match the Wobbe index of the natural gas it is pumping into its pipelines at that time.

Using similar methods, DME can be mixed with air (or another inert gas, such as nitrogen, carbon dioxide, etc.), in a way that causes the mixture to approximate the Wobbe index of a gas supply. This can allow the DME mixture to be mixed smoothly and “seamlessly” with a gas supply that is being pumped into a certain pipeline, without causing any disruptions in stoves, heaters, furnaces, or other burners that are receiving gas from that pipeline system.

It is believed that DME has never previously been used to supplement natural gas supplies, in gas pipelines. Accordingly, in addition to disclosing a new method for supplementing natural gas in pipelines, this provisional application also discloses new compositions of matter, comprising pressurized mixtures of natural gas, DME, and air or an inert gas, in which the DME, and the air or an inert gas, are mixed in controlled ratios that will match or approximate the Wobbe index of a particular natural gas supply that is being supplemented.

### EXAMPLES

### Example 1

### Making and Cracking MSA

Methods and reagents used to make Marshall's acid and MSA in laboratory conditions, using a batch reactors, have already been described in PCT applications PCT/US03/035396 (published in May 2004 as WO 2004/041399) and PCT/US04/019977, both filed by the same Applicant herein. Therefore, those descriptions will not be repeated herein.

To crack MSA in a manner that releases methanol and SO₂, nitrogen gas (N₂) at a flow rate of 6 to 8 mL/second was passed through a gas bubbler containing 10.0-15.0 g of MSA at 120-140° C. The outlet of the bubbler was connected to a quartz tube with an inner diameter of 2 cm and a length of 20 cm, which (except for short inlet and outlet segments) passed through a furnace In various different tests, the tube was either empty, or a 10 cm length of the tube was loaded with 4 to 8 mesh zeolite beads (Davison Chemicals, code number 54208080237). The outlet of the tube was connected to two bubblers, each containing 5.0 g of D₂O (i.e., water containing the heavier deuterium isotope of hydrogen, for analysis using ₁H-nuclear magnetic resonance) at 4-6° C., for trapping any emerging liquids.

When the tube did not contain zeolite packing, significant quantities of the methyl ester of MSA (a byproduct that was unwanted, in these particular tests) were obtained. However, when zeolite packing was provided in the tubes and the furnace was run at 385° C., the yield of methanol increased greatly, and reportedly approached 100%.

### Example 2

### Synthesis of Ethylene and Liquid Alkanes on Hydroxylated Silicate Monolith

The Applicant purchased (from Vesuvius Hi-Tech Ceramics) the same type of “low surface area reticulated silica monolith” described in Barteau 1996, and processed an MSA preparation (purchased from Aldrich Chemical) on it, using reflux temperatures for several hours. Analysis of the gases that emerged from the refluxing liquid, using ¹H-NMR, ³C-NMR, and gas chromatography, indicated that the gases contained ethylene, and liquid alkanes.

The presence of those compounds in those gases indicated that: (i) when MSA is processed on a suitable activated surface, it can pass through intermediates that will create olefins (such as ethylene) and higher alkanes; (ii) the postulated mechanisms and molecular rearrangements described herein have received experimental support; and, (iii) methods for creating olefins and alkanes from MSA can indeed be provided, by one or more pathways that apparently use MSA anhydride intermediates, apparently including sulfene.

### Example 3

### Decomposition of MSA Outer Anhydride

The Applicant purchased the MSA “outer anhydride” compound, in crystalline form, from Aldrich Chemical. In a reaction beaker, it was heated until the crystals melted and then began to form a clear liquid over a black solid. The liquid and the solid were analyzed, using ¹H-NMR, ¹³C-NMR, and gas chromatography. The results indicated that the clear liquid consisted mainly of MSA and cycloalkanes. The black solid was found to contain cyclic hydrocarbons, naphthenics, and a relatively high quantity of aromatic structures. Some of the aromatic rings were bridged by sulfonate or methylene bridges, and some of the aromatic rings had cyclopropane rings attached to them.

Those results provide experimental support for various postulated mechanisms and molecular rearrangements described herein, and confirm that methods for creating olefins, alkanes (including cycloalkanes), and aromatics from MSA can be provided, by one or more pathways that apparently use MSA anhydride intermediates.

### Example 4

### Tungsten Catalyst for Converting MSA to Ethylene

A conventional silica disc (purchased from the Vesuvius company, Alfred, N.Y.) was used, having a monolith configuration with essentially linear and parallel flow channels, with a diameter of about 1 inch and a thickness of about ½ inch, and a weight of 1.8927 grams. It was immersed in a 5% solution of ammonium tungstate, (NH₃)₂WO₂, in distilled water 15 minutes, giving it a wet weight of 5.5667 grams. It was dried in an oven at 110° C. for 90 minutes, and the dried weight was 2.0676 grams. The immersion and drying process was repeated two more times, using 60 minute drying times, leading to successive wet and dry weights of 5.6744 g, 2.5106 g, 5.8603 g, and 2.2670 g. After the third drying operation was completed, a white powdery residue was present in the bottom of the drying dish; this suggested that the disc may have been saturated. It is generally presumed that all or nearly all of the ammonium emerged from the disc in vapor form during the drying periods, and the additional dry weight was due primarily to tungsten oxide on the surfaces of the silica flow channels.

Following a procedure suggested by Barteau et al in their reports of ketene synthesis, the disc was then “silanized” as follows. 10.5 ml of tetraethyl-orthosilicate (TEOS, purchased from Aldrich Chemical) was put in a 100 ml beaker, and a mixture of 10 ml distilled water containing 6 ml 37% HCl was added. The mixture comprised two distinct layers, visible in the clear beaker. It was heated to 70° C., at which time the boundary layer began to disappear, indicating the formation of a single-phase gel that was slightly cloudy. As the mixture began to gel, the tungstate-treated disc was placed in the liquid. The temperature was sustained at 120° C. for 3 hours, then the disc was removed. Excess gel was scraped from the surfaces of the disc. The wet weight was 6.1611 grams.

Since the results disclosed herein are only preliminary, based on initial testing in a small contract lab that does not have extensive analytical equipment, it is not yet known exactly what effect the TEOS treatment had on the silica-tungstate surfaces of the monolith (e.g., in terms of what types of chemical moieties were added to silicon, oxygen, or tungsten atoms on the support surfaces, or the density of such moieties), or whether the TEOS treatment step was necessary or beneficial for enabling or increasing the yields of ethylene from the reaction.

The disc was dried at 170° C. for 16 hours. Its weight was 2.6033 grams. It was designated as disc 0401-170-1, and was calculated to contain 14.5% of the tungstate residue (presumably tungsten oxide, with little or no ammonium), and 12.9% added material from the TEOS treatment.

### Example 5

### Treatment of MSA with Tungsten Catalyst

The silicate disc, coated with tungsten and silanized as described in Example 4, was wrapped in a thin-layer glass cloth (to form a seal comparable to a gasket) and pushed into a reactor tube. It was heated from a starting temperature of 9° C. to a maximum operating temperature of 344° C., while helium flowed through the reactor at 4 liters/hr. The MSA feed pot was heated from 34° C. to 210° C. over the same span of time.

Ethylene was formed at 344° C. However, its concentration fell with time, as the temperature was increased. When the temperature was decreased back to 344° C., no more ethylene was formed, indicating that the activity of the catalyst had been lost.

At the best operating conditions in the best runs, ethylene comprised 95% of total gaseous hydrocarbons that were released, with the balance apparently being methane, as determined by gas chromatography. A relatively small quantity of liquid (apparently methanol) was also recovered in a liquid trap.

Thus, there has been shown and described a new and useful means for synthesizing higher alkanes from methane, via pathways that involve MSA and MSA anhydrides, and there have also been disclosed various additional enhancements in this system. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Basickes, N., et al, J Am Chem Soc 118: 13111 (1996)
Blavins, J. J., et al, J Org Chem 66: 4285 (2001)
Broekhuis, R. R., et al, Catalysts Today 69: 887 (2001)
Chuang, T. J., et al, J Electron Spectroscopy & Related Phenomena 98-99: 149 (1999)
Danon, A., et al, Rev Sci Instrum 58: 1724 (1987)
Dubois, D. R., et al, Fuel Proc. Technology 83: 203 (2003)
Dunn, J. P., et al, Applied Catalysis B: Environmental 19: 103 (1998)
Gesser, H. D., et al, Chem Rev 85: 235 (1985)
Giakoumelou, I., et al, Catalysis Letters 78: 209 (2002)
Gilbert, G. E., Sulfonation and Related Reactions (Interscience Publishers, 1965)
Gilson, T. R., J Solid State Chemistry 117: 136 (1995)
Hayes, R. E., et al, Introduction to Catalytic Combustion (Gordon & Breach Science Publ., Amsterdam, 1997)
Keil, F. J., Microporous and Mesoporous Materials 29: 49 (1999)
Lefferts, L., et al, Applied Catalysis 23: 385 (1986)
Li, P., et al, Surface Science 380: 530 (1997)
Li, A-H, et al, Chem Rev 97: 2341 (1997)
Lie, L. H., et al, J Phys Chem B 106: 113 (2002)
Liu, W., AIChE Journal 48: 1519 (2002)
Liu, W., et al, Ind Eng Chem Res 41: (2002)
Liu, Z. and Liang, J., Current Opinion in Solid State and Materials Science 4: 80 (1999)
Liu, H., et al, J Catalysis 208: 1 (2002)
Lobree, L. J., et al, Ind Eng Chem Res 40: 736 (2001)
Mukhopadhyay, S., et al, Angew Chem Int Ed 42: 2990 (2003)
Mukhopadhyay, S., et al, Amer Chem Soc 2002: A-E (2002)
Muller, A., et al, Catalysis Today 81: 337 (2003)
Olah, G. A., et al, J Amer Chem Soc 106: 2143 (1984)
Olah, G. A., Acc Chem Res 20: 422 (1987)
Peng, X-D., et al, Rev Sci Instrum 63: 3930 (1992)
Periana, R. A., et al, Science 259: 340 (1993)
Periana, R. A., et al, Chem Commun 2002: 2376 (2002)
Periana, R. A., et al, Science 280: 560 (1998)
Porcelli, R. V., et al, Hydrocarbon Proc., March 1986: 37 (1986)
Raja, L. L., et al, Catalysis Today 59: 47 (2000)
Romm, L., et al, J Phys Chem A 105: 7025 (2001)
Sagawa, K., et al, J Catalysis 131: 482 (1991)
Sassi, A., J Phys Chem B 106: 8768 (2002)
Stöcker, M., Microporous Mesoporous Materials 29: 3 (1999)
Tatibouet, J. M., et al, J Catalysis 161: 873 (1966)
Tatibouet, J. M., et al, Applied Catalysis 148: 213 (1996)
te Velde, G., et al, J Comput Chem 22: 931 (2001)
Won, T-J., et al, Inorganic Chemistry 34: 4499 (1995)
Zhai, R-S., et al, preprint downloaded from American Chemical Society website (Langmuir), 2004
Zhou, X., et al, Chem Commun: 2294 (2003)

1. A method of making liquid alkanes, comprising the following steps:
a. reacting methanesulfonic acid with a dehydrating agent, under conditions that create a methanesulfonic acid anhydride;
b. reacting the methanesulfonic acid anhydride in a manner that creates a liquid alkane preparation having a purity sufficient for use as fuel.

2. The method of claim 1 wherein the methanesulfonic acid anhydride comprises sulfene, and wherein the sulfene is reacted with at least one second compound under conditions that allow the sulfene to transfer at least one methylene group into the second compound.

3. A method of making at least one olefin, comprising the step of reacting methanesulfonic acid with a dehydrating agent, under conditions that create sulfene as a reactive intermediate, and allowing the sulfene to react with itself in a manner that forms at least one olefin.

4. The method of claim 3 wherein the olefin comprises ethylene.

5. A method of making at least one olefin, comprising the step of reacting methanesulfonic acid with a catalytic surface comprising metal atoms.

6. The method of claim 5 wherein at least some metal atoms on the catalytic surface are in a +6 oxidation state.

7. The method of claim 5 wherein at least some metal atoms on the catalytic surface are selected from the group consisting of tungsten, vanadium, and ruthium.

8. A method of making at least one olefin, comprising the step of contacting sulfene with a catalytic surface comprising metal atoms.

9. The method of claim 8 wherein at least some metal atoms on the catalytic surface are in a +6 oxidation state.

10. The method of claim 8 wherein at least some metal atoms on the catalytic surface are selected from the group consisting of tungsten, vanadium, and ruthium.

11. A method of making cyclopropane, comprising the step of reacting methanesulfonic acid with a dehydrating agent under conditions that create sulfene as a reactive intermediate, and allowing the sulfene to react with itself in a manner that forms ethylene, and allowing the sulfene to react with ethylene in a manner that forms cyclopropane.

12. A method of making sulfene, comprising the step of treating methanesulfonic acid with a catalytically active surface that promotes formation of sulfene from methanesulfonic acid.

13. The method of claim 12, wherein the catalytically active surface comprises metal atoms selected from the group consisting of tungsten, vanadium, and ruthium.

14. A method of making methanesulfonic acid, comprising the following steps:
a. cleaving di(methyl-sulfonyl) peroxide in a manner that releases two methanesulfonic acid radicals; and,
b. contacting said methanesulfonic acid radicals with methane, in a reaction mixture that also contains sulfur trioxide.

15. The method of claim 14, wherein said di(methyl-sulfonyl) peroxide is formed by electrolysis of methanesulfonic acid.

16. A method of making dimethyl ether, comprising the steps of:
a. condensing methanesulfonic acid with methanol, under reaction conditions that form at least one condensation product; and,
b. reacting at least one of said condensation products in a manner that causes it to release dimethyl ether.

17. The method of claim 16, wherein at least one of said condensation products comprises methyl-methanesulfonate ester.
